(19) United States
(12) Patent Application Publication
(10) Pub. No.: US 2026/0146091 A1
(43) Pub. Date: May 28, 2026

(54) NANOBODIES FOR CANCER THERAPY

(71) Applicants: FUNDACIÓN PARA LA INVESTIGACIÓN MÉDICA APLICADA, PAMPLONA (ES); UNIVERSIDAD DE LA REPÚBLICA (UDELAR), MONTEVIDEO (UY)

(72) Inventors: NOELIA ROMINA SILVA PILIPICH, PAMPLONA (ES); CRISTIAN SMERDOU PICAZO, PAMPLONA (ES); LUCIA MARÍA VANRELL MAJÓ, PAMPLONA (ES); GUALBERTO GONZÁLEZ SAPIENZA, MONTEVIDEO (UY)

(21) Appl. No.: 19/122,051

(22) PCT Filed: Oct. 19, 2023
(86) PCT No.: PCT/EP2023/079157

§ 371 Date: Apr. 17, 2025

(30) Foreign Application Priority Data
Oct. 20, 2022 (EP) 22383011.8

(51) Int. Cl. C07K 16/28; A61K 38/20; A61K 47/68; A61P 35/00; C12N 15/86

(52) U.S. Cl. CPC C07K 16/2827; A61K 38/208; A61K 47/6813; A61P 35/00; C12N 15/86; C07K 2317/52; C07K 2317/565; C07K 2317/569; C07K 2317/76; C07K 2317/92; C12N 2770/36143

(57) ABSTRACT

The present disclosure provides single domain antibodies (VHH or nanobodies) and conjugates thereof which are able to inhibit binding between PD-L1 and PD-1 with high efficiency. The disclosure additionally refers to viral vectors comprising said nanobodies or nanobody conjugates, and use of said nanobodies, nanobody conjugates and vectors for use in treating cancer.

This application claims the benefit of European Patent Application EP22383011.8 filed on 20 Oct. 2022.

### TECHNICAL FIELD

The present disclosure is related with the field of cancer therapy, in particular, with the development of new immune checkpoint inhibitors for the treatment of cancer.

### BACKGROUND ART

Tumor cells use many strategies to evade the immune system, such as engaging immune checkpoint (IC) pathways that induce immunosuppressive functions. Among the different IC receptors, T-lymphocyte-associated protein 4 (CTLA-4) and programmed cell death protein 1 (PD-1) are being used as therapeutic targets in different cancers. Several monoclonal antibodies (mAbs) blocking these IC pathways (named IC inhibitors, or ICIs) have been shown to elicit a powerful antitumor effect that translates into significant and lasting clinical responses in a small fraction of patients. Antibodies able to block the PD-1/PD-L1 axis, such as nivolumab, pembrolizumab, and atezolizumab, are showing remarkable therapeutic effects in patients with different types of tumors [Xiang Z, et al, Front Pharmacol. 2022]. However, frequent adverse effects observed in patients treated with checkpoint inhibitors, as well as the lack of responses in some tumor types, makes necessary the improvement of these therapies [Sun G, et al, Int J Oncol. 2022].

Durable clinical benefits of these agents are mainly limited because of primary resistance to these therapies or acquired resistance after an initial response. Furthermore, because the receptors constitute a natural mechanism to maintain self-tolerance, the systemic administration of ICIs results in an immune-based attack on normal tissues in a non-negligible fraction of patients. These immune-related adverse events (irAEs) can potentially affect every organ of the body, being dermatitis and thyroiditis the most common ones, followed by others of major concern, such as pneumonitis, hepatitis colitis, hepatitis, hypophysitis, nephritis, myositis, and adrenalitis.

Therefore, there is an evident need to generate safer and more effective IC inhibitors for the treatment of cancer, ideally, in a higher percentage of patients.

### SUMMARY OF INVENTION

The present inventors have developed new IC inhibitors that overcome many of the drawbacks mentioned above. In particular, the inventors have developed single domain antibodies (herein also referred to as VHH or nanobodies) that are able to inhibit the interaction of Programmed Cell Death Protein 1 (PD-1) and Programmed Death Ligand-1 (PD-L1) for human and mouse orthologs.

Thus, the first aspect of the disclosure relates to a single domain antibody comprising the following complementary determining regions (CDRs): (i) CDR1 as set forth by SEQ ID NO: 1, CDR2 as set forth by SEQ ID NO: 2 and CDR3 as set forth by SEQ ID NO: 3; or (ii) CDR1 as set forth by SEQ ID NO: 5, CDR2 as set forth by SEQ ID NO: 6 and CDR3 as set forth by SEQ ID NO: 7.

As shown in the examples below, the nanobody comprising the CDRs of SEQ ID NO: 1, SEQ ID NO: 2, and SEQ ID NO: 3 binds to PD-L1, and is therefore an anti-PD-L1 nanobody, while the nanobody comprising the CDRs of SEQ ID NO: 5, SEQ ID NO: 6, and SEQ ID NO: 7 binds to PD-1, and is therefore an anti-PD-1 nanobody. These novel anti-PD-1 and anti-PD-L1 nanobodies are shown to block the binding between human PD-L1 and PD-1. The inhibitory activity of the nanobodies described herein is surprising, since the nanobodies only contain three (and not six) CDRs for antigen binding. The examples below additionally show that this inhibitory activity translates into antitumor effect in MC38 colon adenocarcinoma tumors.

Moreover, the disclosed nanobodies have several advantages compared to known anti-PD-L1 and anti-PD-1 antibodies. For example, the nanobodies of the first aspect show cross-reactivity for mouse and human PD-L1 and PD-1, and inhibit the binding of PD-1 with PD-L1 of both species. Cross-reactivity is a desirable feature for translational research, allowing for a smoother translation of animal studies into a clinical set-up. A further advantage is that, due to their smaller size, these nanobodies enter and are distributed more efficiently in tumors. They also have advantages in terms of manufacture, as they are easier and more cost-effective to produce. Nanobodies have been successfully expressed in different systems obtaining high yields, including bacteria, yeast, plant cells, insect cells, and mammalian cells. This is possible thanks to their high solubility and physicochemical stability, single-domain nature, and the fact that posttranslational modifications are not needed. The possibility of using inexpensive systems such as E. coli is a great advantage for cost-effective industrial scale-up. The nanobodies are also easier to handle for preparing pharmaceutical or diagnostic formulations.

A second aspect of the present disclosure refers to a nanobody conjugate comprising a nanobody as defined in the first aspect.

The inventors have found that, although monomeric nanobodies already showed antitumor effects comparable to known antibodies, the fusion of the nanobodies to another molecule forming nanobody conjugates provides several advantages. For example, conjugating a nanobody as defined above with an immunoglobulin G (IgG) fragment crystallizable (Fc) surprisingly improved the PD-1/PD-L1 inhibition profile. This is evidenced in the examples of the present disclosure, which show that conjugation of two copies of the same nanobody to an immunoglobulin G fragment crystallizable (Fc) domain surprisingly improved the PD-1/PD-L1 inhibition profile and promoted a more potent antitumor activity in a mouse model of colorectal cancer compared to conventional antibodies. In this sense, the examples below show that homo-dimerization of the nanobodies of the first aspect using an IgG Fc domain resulted in approximately 8- and 40-fold IC50 reduction in PD-1/PD-L1 binding assays, for the anti-PD-L1 and anti-PD1 nanobodies, respectively.

In another example, it was further found that a nanobody conjugate comprising IL-12 (a cytokine with potent antitumor activity) and one of the nanobodies described herein presents a strong synergy. Without wishing to be bound by theory, the inventors think this may be because IL-12 induces the production of IFN-gamma, which in turn induces the expression of PD-L1, which can limit the activity of the cytokine. Advantages of these nanobody-cytokine nanobody conjugates (also herein referred to as immunocytokines or ICKs), would be: (i) synergistic effects on the action of IL-12 and anti-PD-1 or anti-PD-L1 nanobodies, (ii) concentration of IL-12 activity in the tumor due to PD-L1 and PD-1 high levels in tumors that, by interacting with the nanobody moiety, would facilitate ICK retention in tumor tissue, and (iii) reduction of the possible toxicity of IL-12, as it will be retained in the tumor tissue.

It was also found that the nanobodies and nanobody conjugates defined above could be successfully delivered using a replication-defective alphavirus vector based on Semliki Forest virus (SFV). As shown in the examples below, delivery of the nanobodies described herein through a SFV vector not only enabled a high and local intratumoral nanobody or nanobody conjugate expression but also elicited an important and co-adjuvant response which synergized with the nanobodies' immune-stimulatory and antitumoral activity. Delivery of the nanobodies through viral vectors provides further advantages in the manufacturing process and regulatory requirements for clinical translation. Moreover, a remarkable feature is that a single dose of the vector expressing locally the dimerized nanobodies for a very short period of time was able to promote potent and long-lasting antitumor responses.

SFV-based RNA viral vectors additionally show several advantages over other vectors for cancer treatment, such as higher expression levels, broad tropism, induction of immunogenic apoptosis in tumor cells, and the ability to elicit powerful IFN-I responses.

Thus, a third aspect of the present disclosure refers to a polynucleotide encoding for a nanobody as defined in the first aspect or a nanobody conjugate as defined in the second aspect, and a fourth aspect refers to an expression vector comprising the polynucleotide as defined in the third aspect.

A fifth aspect of the present disclosure refers to a host cell comprising a polynucleotide as defined in the third aspect or an expression vector as defined in the fourth aspect.

A sixth aspect refers to a viral particle comprising a polynucleotide as defined in the third aspect or an expression vector as defined in the fourth aspect.

A seventh aspect provides a method for producing nanobodies or nanobody conjugates as defined in the first aspect and second aspects, or for producing viral particles as defined in the sixth aspect, said method comprising the steps of:
(a) culturing a host cell according to the fifth aspect under conditions suitable for producing the nanobodies, nanobody conjugates, or viral particles, thereby obtaining a culture containing said nanobodies, nanobody conjugates, or viral particles; and
(b) isolating or recovering said nanobodies, nanobody conjugates, or viral particles from said culture.

An eight aspect refers to a pharmaceutical composition comprising a nanobody, or a nanobody conjugate, or an expression vector, or a host cell, or a viral particle, all of them as defined in the aspects above, together with pharmaceutically acceptable excipients and/or carriers.

The disclosure also provides, in the ninth aspect, a pharmaceutical composition, or a nanobody or a nanobody conjugate, or an expression vector, or a host cell, or a viral particle, all of them as defined in the aspects above, for use in therapy. This aspect may be reworded as use of a pharmaceutical composition, or a nanobody or a nanobody conjugate, or an expression vector, or a host cell, or a viral particle, all of them as defined in the aspects above, for the preparation of a medicament. Also disclosed is a method of treatment which comprises administering to a subject in need thereof a pharmaceutical composition, or a nanobody or a nanobody conjugate, or an expression vector, or a host cell, or a viral particle, all of them as defined in the aspects above.

A tenth aspect provides a pharmaceutical composition, or a nanobody or a nanobody conjugate, or an expression vector, or a host cell, or a viral particle, all of them as defined in the aspects above, for use in treating cancer. This aspect may be reworded as use of a pharmaceutical composition, or a nanobody or a nanobody conjugate, or an expression vector, or a host cell, or a viral particle, all of them as defined in the aspects above, for the preparation of a medicament for treating cancer. Also disclosed is a method for treating cancer which comprises administering to a subject in need thereof a pharmaceutical composition, or a nanobody or a nanobody conjugate, or an expression vector, or a host cell, or a viral particle, all of them as defined in the aspects above.

The eleventh aspect of the present disclosure provides for use of a nanobody or a nanobody conjugate according to the first and second aspects:
(i) for detecting human PD-L1 and/or PD-1 molecules;
(ii) for flow cytometry assay;
(iii) for cell immunofluorescence detection; or
(v) for diagnosing cancer.

The twelfth aspect of the present disclosure provides a method for detecting PD-L1 and/or PD-1 protein in a sample, and said method comprises the steps of:
(1) contacting the sample with a nanobody or a nanobody conjugate according to the first and second aspects;
(2) detecting the antigen-antibody complex, wherein the detected complex indicates the presence of PD-L1 protein or PD-1 protein.

Finally, a thirteenth aspect refers to a diagnostic agent comprising a nanobody or a nanobody conjugate according to the first and second aspects.

### BRIEF DESCRIPTION OF DRAWINGS

FIG. 1. Expression of anti-PD-1 and PD-L1 nanobodies from SFV vectors in vitro and antitumor activity in vivo. (A) Diagram of SFV vectors encoding monomeric nanobodies (Nb), showing the protein product on the right (not to scale). The subgenomic promoter (sgPr) that allows the transcription of the subgenomic RNA encoding the Nb is shown. (B-C) BHK-21 cells were infected with SFV Viral particles expressing the indicated transgenes at MOI 20, or mock infected, and analyzed at 24 h by Western blot, using anti-HA antibody (B), and specific PD-1 or PD-L1 binding ELISA for quantification (the percent of each fraction is indicated above bars) (C). (D and E) The antitumor activity of SFV vectors encoding monomeric anti-PD-1 (SFV-Nb11) (D) and PD-L1 (SFV-Nb6p) (E) nanobodies was evaluated in the MC38 subcutaneous tumor model using SFV-LacZ and saline as controls. Mice received one dose of 3×10⁸ viral particles intratumorally. Left graphs, tumor growth after treatment. Data represent mean±SEM (n=7 per group), a representative experiment out of two with similar results is shown. Right graphs, survival curves of treated animals. UTR, untranslated region; HA, hemagglutinin tag; S, supernatant; CE, cell extract; ns, not significant. Magnification in B, 400×.

FIG. 2. Expression of nanobody-Fc fusion proteins from SFV vectors in vitro compared to conventional antibodies. (A) Schematic diagrams of SFV vectors encoding nanobodies (Nb) against PD-1 and PD-L1 fused to the indicated mouse IgG (mIgG) Fc domains (SFV-Nb11-Fc and SFV-Nb6p-Fc, respectively) and conventional full-length antibodies (mAb) against mouse PD-1 and PD-L1 (SFV-aPD1 and SFV-aPDL1, respectively). On the right side, schematic diagrams of the different antibodies and their estimated molecular weight are shown. (B-C) BHK-21 were infected with SFV Viral particles expressing the indicated transgenes at MOI 20, or mock infected, and analyzed at 24 h by specific IgG ELISA for quantification (the percent of each fraction is indicated above bars) (B), and Western blot using antibodies against mouse IgG, SFV replicase, and α-actin (C). UTR, untranslated region; DTT, dithiothreitol; HC, IgG heavy chain. Magnification in B, 400×.

FIG. 3. Inhibition of PD-1/PD-L1 binding in vitro. Inhibition curves were performed using purified nanobodies against PD-1 (A) and PD-L1 (B). Monomeric nanobodies, nanobodies fused to Fc domains, or commercially available antibodies were included in each assay. Mouse (left) or human (right) PD-1/PD-L1 ectodomains were used. Data represent mean±SD of the percentage of PD-1/PD-L1 binding, considering wells with no blocking antibody as 100% of binding.

FIG. 4. Antitumor activity of SFV vectors encoding nanobody-Fc fusion proteins. The antitumor activity of SFV vectors expressing nanobodies or mAbs against PD-1 (A) and PD-L1 (B) was tested in the MC38 subcutaneous tumor model. When tumors reached a size of approximately 20 mm³, a single intratumoral dose of 3×10⁸ Viral particles of the indicated vectors was administered. SFV-LacZ and saline were used as controls. In A and B: left graphs, tumor growth evolution (mean±SEM, a representative experiment out of two with similar results is shown, n=6-8 per group); middle graphs, survival (pooled data from two independent experiments, n=13-15 per group); right graphs, tumor growth evolution in cured mice rechallenged after 2-3 months with 5×10⁵ MC38 cells in the left flank. Naïve untreated mice were included as controls. *, p<0.05; **, p<0.01; ****, p<0.0001; ns, not significant.

FIG. 5. Expression of nanobodies in vivo from SFV vectors administered intratumorally. Mice bearing ˜40 mm³ subcutaneous MC38 tumors were injected intratumorally with 3×10⁸ SFV Viral particles encoding monomeric or Fc-fused nanobodies (n=4-5 per group). Mice were sacrificed one or five days later to evaluate nanobody expression in vivo. (A) Nanobody levels in tumors normalized by total protein content. (B) Nanobody levels in serum. (C) Levels of monomeric Nb11 and Nb6p nanobodies in urine samples. (D) Levels of Nb11-Fc in urine. *, p<0.05; ****, p<0.0001; ns, not significant.

FIG. 6. Characterization of the immune cell infiltration in MC38 tumors treated with SFV vectors encoding Nb6p-Fc. Mice bearing ˜50 mm³ MC38 tumors (n=5-6/group) received one intratumoral dose of 3×10⁸ SFV Viral particles, and five days later they were sacrificed to analyze the immune cell infiltrate by flow-cytometry. (A) Tumor growth curves after treatment and tumor weight at sacrifice (day 5 after treatment). (B-D) Analysis of the CD8⁺ T cell population in tumor samples. (B) CD8⁺ T cell infiltration and expression of activation markers. (C) Analysis of MuLV-specific CD8⁺ T cells. (D) PD-1 expression on CD8+ T cells and MuLV-specific CD8⁺ T cells. (E) Analysis of different innate immune cell populations in tumor samples. Asterisks above bars indicate comparison to saline group. One sample from SFV-LacZ and one from SFV-Nb6p-Fc were excluded from this analysis due to low viability of the samples. *, p<0.05; **, p<0.01; ***, p<0.001.

FIG. 7. Antitumor activity of a DNA/RNA layered SFV vector expressing Nb11-Fc delivered by electroporation. (A) Schematic representation of pBK-SFV DNA plasmid vector harboring under the CMV promoter the SFV replicon expressing Nb11-Fc (pBK-SFV-Nb11-Fc). (B) Expression of Nb11-Fc from pBK-SFV plasmid was confirmed in vitro by Western blot from transfected BHK-21 cells, including pBK-SFV-LacZ and pBK-SFV-Nb6p-Fc in the analysis. (C) Experimental design used for delivery of pBK-SFV plasmids into tumors. Mice bearing ˜20 mm³ MC38 subcutaneous tumors were injected intratumorally at the indicated times with 20 μg of pBK-SFV plasmids and electroporated as described in Materials and Methods. (D) Tumor growth curves. Data represent mean±SEM (n=6-7). Treatment days are indicated by arrows. (E) Survival curves for the treated animals. (F) Survival after tumor rechallenge in animals that had complete remissions after treatment. Naïve untreated mice were included as controls. *, p<0.05; ****, p<0.0001; ns, not significant.

FIG. 8. Characterization of anti-PD-L1 and anti-PD-1 nanobodies. (A-D) The ability of the anti-PD-L1 and anti-PD-1 nanobodies to inhibit the interaction between PD-1 and PD-L1 from human and mouse was evaluated by ELISA, using commercial PD-L1 ectodomains fused to human IgG1 Fc. (A) Human PD-1/PD-L1 binding inhibition of different nanobodies used at 3.3 nM. Control anti-hPD-L1 monoclonal antibody (aPDL1 mAb) was used at 66 nM. (B) Inhibition curves of human PD-1/PD-L1 binding for the best nanobodies selected in (A). (C) Mouse PD-1/PD-L1 binding inhibition of different nanobodies at 300 nM. Control anti-mPD-L1 monoclonal antibody (aPDL1 mAb) was used at 130 nM. (D) Inhibition curves of mouse PD-1/PD-L1 binding for the best nanobodies selected in (C). In A-D, data represent percentage of binding inhibition, considering 100% of binding the condition without antibody. Ctrl Nb, control nanobody.

FIG. 9. Antitumor activity of SFV vectors encoding nanobody-Fc fusion proteins in the B16OVA tumor model. Mice were injected with 5×10⁵ B16OVA cells in the right flank. When tumors reached a size of approximately 20 mm³, they were treated intratumorally with 3×10⁸ Viral particles of the indicated SFV vectors or saline. A) Tumor growth after treatment. Data represent mean±SEM, n=9 per group. B) Survival curves for animals treated with the different vectors. **, p<0.01; ***, p<0.001; ****, p<0.0001.

FIG. 10. Abscopal effect induced by SFV-Nb11-Fc vector. Mice bearing two subcutaneous contralateral MC38 tumors were treated by injecting the right flank tumor with two doses of 3×10⁸ Viral particles of SFV-Nb11-Fc or saline on days 0 and 5. (A) Tumor growth evolution of treated (continuous line) or untreated (dashed line) tumors. Data represent mean±SEM, n=6-7. B) Survival curves. *p<0.01; ****, p<0.0001; ns, not significant.

FIG. 11. Immune cell characterization in draining lymph nodes from MC38 tumors treated with SFV vectors encoding Nb-Fc. Mice bearing ˜50 mm³ MC38 tumors (n=5-6/group) received one i.t. dose of 3×10⁸ SFV Viral particles, and five days later they were sacrificed to analyze the immune cell infiltrate in the tumor draining lymph node (dLN) by flow-cytometry. (A) Analysis of CD8⁺ T cells. (B) Analysis of the myeloid population (CD11b⁺ cells). Asterisks above bars indicate comparison to saline group. One sample from SFV-LacZ and one from SFV-Nb11-Fc were excluded from analysis in panel A due to low viability of the samples. *, p<0.05; **, p<0.01; ***, p<0.001, ****, p<0.0001.

FIG. 12. Diagram of SFV vectors expressing ICKs. Diagram of SFV vectors encoding nanobodies (Nb) fused to either scIL 12 (A) and dcIL 12 (B), showing the protein product on the right (not to scale). The subgenomic promoter (sgPr) that allows the transcription of the subgenomic RNA encoding the Nb is shown by an arrow.

FIG. 13. Analysis of ICK expression. (A) BHK-21 cells were infected with SFV Viral particles expressing the indicated transgenes at a multiplicity of infection (MOI) of 10, or mock infected, and analyzed at 24 h by specific mouse IL-12 ELISA for quantification. (B) To measure the activity of IL-12 and ICKs supernatants from cells infected with SFV-ICK Viral particles were added to splenocytes from healthy mice, incubated for 48 h and quantified by commercial IFNγ-specific ELISA. sc, scIL 12; dc, dcIL 12.

FIG. 14. Antitumor effect of SFV vectors expressing ICKs. The antitumor activity of SFV vectors expressing ICKs was tested in the MC38 subcutaneous tumor model. When tumors reached a size of approximately 20 mm³, a single intratumoral dose of 2×10⁷ Viral particles of the indicated vectors was administered. Saline was used as negative control. (A) Tumor growth evolution (mean±SEM, n=7-8 per group). (B) Survival (numbers indicate: complete regressions/number of mice). (C) IL-12 levels in serum 24 h after treatment. (D). Body weight evolution.

FIG. 15. Dose range finding study for SFV-dcIL 12-Nb11 and SFV-dcIL 12. The antitumor activity of SFV-dcIL 12-Nb11 and SFV-dcIL 12 was compared in the MC38 subcutaneous tumor model. When tumors reached a size of approximately 20 mm³, a single intratumoral dose of the indicated vectors at the indicated doses was administered. Saline was used as negative control. (A) Tumor growth evolution (mean±SEM, n=7 per group). (B) Survival (numbers indicate: complete regressions/number of mice). *, p<0.05; **, p<0.01; ****, p<0.0001; ns, not significant.

### DETAILED DESCRIPTION OF THE INVENTION

Unless defined otherwise, all technical and scientific terms used herein have the same meaning as is commonly understood by one with skill in the art to which this invention belongs at the time of filling. However, in the event of any latent ambiguity, definitions provided herein take precedent over any dictionary or extrinsic definition. Further, unless otherwise required by context, singular terms shall include pluralities and plural terms shall include the singular.

As used herein, the indefinite articles “a” and “an” are synonymous with “at least one” or “one or more.” Unless indicated otherwise, definite articles used herein, such as “the” also include the plural of the noun.

### Nanobodies

The first aspect of the present disclosure provides a nanobody selected from (i) an anti-PD-L1 nanobody and (ii) an anti-PD-1 nanobody which comprise the following complementary determining regions (CDRs): (i) CDR1 as set forth by SEQ ID NO: 1, CDR2 as set forth by SEQ ID NO: 2 and CDR3 as set forth by SEQ ID NO: 3; or (ii) CDR1 as set forth by SEQ ID NO: 5, CDR2 as set forth by SEQ ID NO: 6 and CDR3 as set forth by SEQ ID NO: 7.

As used herein, the terms “single domain antibody (VHH)” and “nanobody” are used interchangeably and refer to an antibody fragment consisting of a single monomeric variable antibody domain. It is the smallest antigen-binding fragment with complete function. Structurally, nanobodies are similar to the heavy chain variable domain of conventional antibodies, with three hypervariable complementary determining regions (CDRs) interspersed with four regions that are more conserved, termed framework regions (FRs).

Therefore, the present invention also includes fragments, derivatives and analogs of the nanobodies. As used herein, the terms “fragment,” “derivative,” and “analog” refer to a polypeptide that substantially retains the same biological function or activity of a nanobody of the invention. Polypeptide fragments, derivatives or analogs of the invention may be (i) polypeptides having one or more conservative or non-conservative amino acid residues substitutions; or (ii) a polypeptide having a substituent group in one or more amino acid residues; or (iii) a polypeptide formed by fusing any of the above and another compound (see below for nanobody conjugates).

The nanobody of the present invention refers to a polypeptide including the above CDR regions having PD-L1 or PD-1 protein binding activity. For example, the present disclosure includes not only intact nanobodies but also fragments of active nanobody or nanobody conjugates formed from nanobodies with other sequences (see below for nanobody conjugates). The term also encompasses variant forms of polypeptides comprising the above CDR regions that have the same function as the nanobodies of the invention but may differ in their sequence. These variations include, but are not limited to, deletion, insertions and/or substitutions of one or several (for example, 1-30, 1-20, or 1-10) amino acids, and addition of one or several (generally less than 20, or less than 10, or less than 5) amino acids at C-terminus and/or N-terminus. For example, in the art, the substitution of amino acids (natural or non-natural amino acids) with analogical or similar properties usually does not alter the function of the protein. In another example, addition of one or several amino acids at the C-terminus and/or N-terminus usually does not change the function of the protein. The contemplated variations also include polypeptides having a substituent group in one or more amino acid residues, as well as homologous sequences, conservative variants, allelic variants, natural mutants, and induced mutants.

In the present disclosure, “a conservative variant” refers to the polypeptides in which there are amino acids substituted by amino acids having analogical or similar properties, compared to the amino acid sequence of the nanobody of the present invention. Usually, substituted amino acids are up to 10, up to 8, up to 5, or most usually up to 3. These conservative variant polypeptides may be produced according to the amino acid substitutions in Table 1.

TABLE 1

| Original residue | Representative substitution | Preferable substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

In one embodiment of the first aspect, the nanobody according to the first aspect blocks the binding between PD-L1 and PD-1 with half maximal inhibitory concentration (IC50) of 6 nm or lower. The “half maximal inhibitory concentration (IC50)” is a measure of the potency of a substance in inhibiting a specific biological or biochemical function. IC50 is a quantitative measure that indicates how much of a particular inhibitory substance (e.g. drug) is needed to inhibit, in vitro, a given biological process by 50%. In the present disclosure, the biological process to be inhibited is the binding between PD-L1 and PD-1. The IC50 can be measured, for instance, by a competitive ELISA in which binding of PD-1 to PD-L1 is measured in the presence of different concentrations of the nanobody to be tested. In particular embodiments, the IC50 of the nanobody is equal or below 5 nM, equal or below 4 nM, equal or below 3 nM, equal or below 2 nM, equal or below 1 nM, equal or below 0.9 nM, equal or below 0.8 nM, equal or below 0.7 nM, equal or below 0.6 nM, equal or below 0.5 nM, equal or below 0.4 nM, equal or below 0.3 nM, equal or below 0.2 nM, or equal or below 0.1 nM.

In one embodiment of the first aspect, the nanobody is humanized. In another embodiment, the nanobody is recombinant. In another embodiment, the nanobody is isolated. The term “humanized” nanobody refers to a nanobody whose peptide sequence has been modified to increase its similarity to antibody variants produced naturally in humans. This can be done replacing one or more amino acid residues in the amino acid sequence of the nanobody sequence (and in particular in the framework sequences) by one or more of the amino acid residues that occur at the corresponding position(s) in a VH domain from a conventional antibody from a human being. The term “recombinant” nanobody refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) expression system, or a non-human cell expression system (e.g., yeast, bacteria, insect), or isolated from a recombinant combinatorial human antibody library. The term “isolated” refers to substances that are at least partially free of other biological molecules from the cells or cell culture from which they are produced, for example, at least partially free of expression system components such as biological molecules from a host cell or of the growth medium thereof, or at least partially free of cell components of the organism (e.g., animal) from which they are derived. Generally, the term “isolated” is not intended to refer to a complete absence of such biological molecules or to an absence of water, buffers, or salts or to components of a pharmaceutical formulation that includes the referred substances.

In one particular embodiment of the first aspect, the nanobody comprises or consists of a sequence having at least 90%, at least 91%, at least 92%, at least 93%, at least 94%, or at least 95% sequence identity with SEQ ID NO: 4. In a more particular embodiment, the nanobody comprises or consists of a sequence having at least 96%, at least 97%, at least 98%, or at least 99% sequence identity with SEQ ID NO: 4. In an even more particular embodiment, the nanobody comprises or consists of a sequence as set forth in SEQ ID NO:4.

In one particular embodiment of the first aspect, the nanobody comprises or consists of a sequence having at least 90%, at least 91%, at least 92%, at least 93%, at least 94%, or at least 95% sequence identity with SEQ ID NO: 8. In a more particular embodiment, the nanobody comprises or consists of a sequence having at least 96%, at least 97%, at least 98%, or at least 99% sequence identity with SEQ ID NO: 8. In an even more particular embodiment, the nanobody consists of a sequence as set forth in SEQ ID NO:8.

In the present invention the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The percentage of identity determines the number of identical residues over a defined length in a given alignment. Thus, the level of identity between two sequences or (“percent sequence identity”) is measured as a ratio of the number of identical positions shared by the sequences with respect to the number of positions compared (i.e., percent sequence identity=(number of identical positions/total number of positions compared)×100). A gap, i.e., a position in an alignment where a residue is present in one sequence but not in the other, is regarded as a position with non-identical residues and is counted as a compared position.

A number of mathematical algorithms for rapidly obtaining the optimal alignment and calculating identity between two or more sequences are known and incorporated into a number of available software programs. For purposes of the present invention, the sequence identity between two amino acid sequences is preferably determined using algorithms based on global alignment, such as the Needleman-Wunsch algorithm (Needleman and Wunsch, J. Mol. Biol. 48:443-453, 1970. DOI: 10.1016/0022-2836 (70) 90057-4), preferably implemented in the Needle program of the EMBOSS package (EMBOSS: The European Molecular Biology Open Software Suite, Rice et al., Trends Genet. 16:276-277, 2000. DOI: 10.1016/s0168-9525 (00) 02024-2); or the BLAST Global Alignment tool (Altschul et al., “Basic local alignment search tool”, 1990, J. Mol. Biol, v. 215, pages 403-410, 1990. DOI: 10.1016/S0022-2836 (05) 80360-2), using default settings. Local alignment also can be used when the sequences being compared are substantially the same length.

### Nanobody Conjugates

As mentioned above, a second aspect refers to nanobody conjugates comprising a nanobody according to the first aspect.

Nanobodies can readily be conjugated to other molecules, such as other proteins or effector domains, to form nanobody conjugates with tailored utility for specific therapeutic applications. The term “nanobody conjugate” refers to a nanobody as defined herein conjugated (joined) to at least a second molecule. The nanobody and the additional molecule(s) are in operable association within the conjugate. Embodiments of this aspect refer to a nanobody conjugate comprising, in addition to one or more nanobodies according to the first aspect, a molecule selected from the group consisting of albumin, a detectable marker, a radionuclide, a drug, a toxin, a polymer, a purification tag, a liposome, a nanoparticle, and combinations thereof. The nanobody may be conjugated (or fused) to the other molecule through the amino or carboxy-terminal end. In some embodiments the nanobody is conjugated to one or more molecules through both the amino or carboxy-terminal ends. All embodiments defined above for the nanobodies of the first aspect also apply to the nanobody conjugates of the second aspect.

In some embodiments the nanobody conjugates are monospecific. In some embodiments the nanobody conjugates are bispecific. In other embodiments the nanobody conjugates are multi-specific. Mono, di or multi-specific as used herein refers to having affinity for one, two, or more antigens, respectively. Specificity may be provided by one or more nanobodies, or other molecules that are able to bind specifically to a particular target.

Immunoconjugates according to the present disclosure also contemplate a nanobody conjugated to at least another nanobody (naked nanobodies). The nanobody and other molecule(s) may be joined directly or through a linker (or spacer). As used herein, “linkers” are peptides of 1 to 50 amino acids length and are typically chosen or designed to be unstructured and flexible. These include, but are not limited to, synthetic peptides rich in Gly, Ser, Thr, Gln, Glu or further amino acids that are frequently associated with unstructured regions in natural proteins. An appropriate linker is, for example, a flexible glycine-serine linker.

In one embodiment, the nanobody conjugate according to the second aspect comprises a nanobody as defined above and at least one further nanobody. In one embodiment, the nanobodies are conjugated in tandem, usually through a linker. For example, tandem repeats of 2, 3, 4, 5, or more nanobodies fused to each other directly or through a linker are disclosed in one embodiment. Configurations other than tandem are possible. In order to increase the in vivo half-life of the nanobody conjugate, the nanobodies may be linked to another molecule, such as albumin.

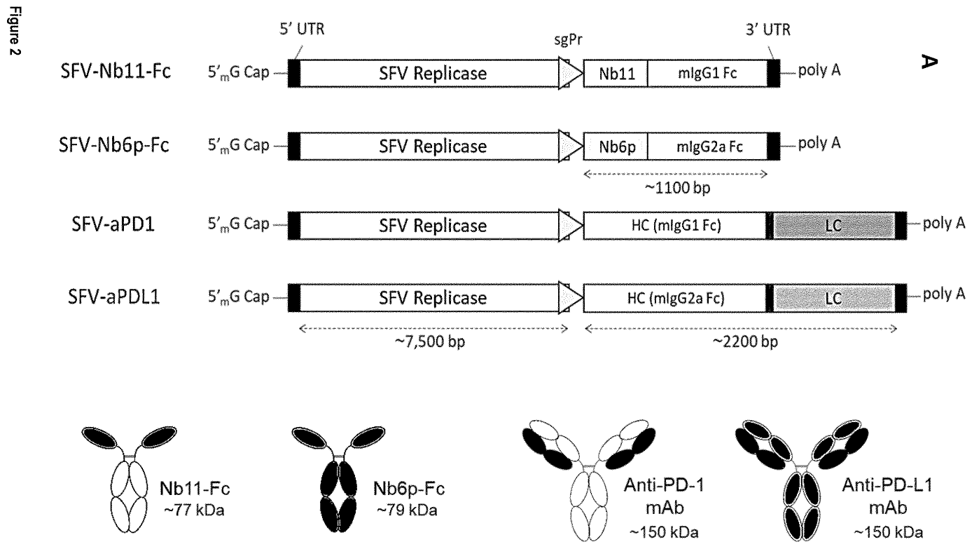

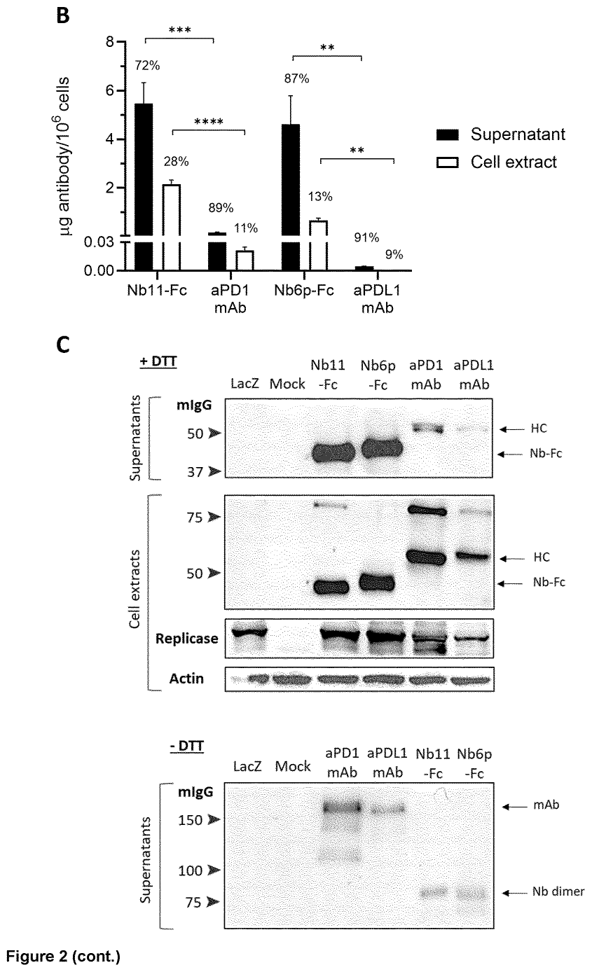

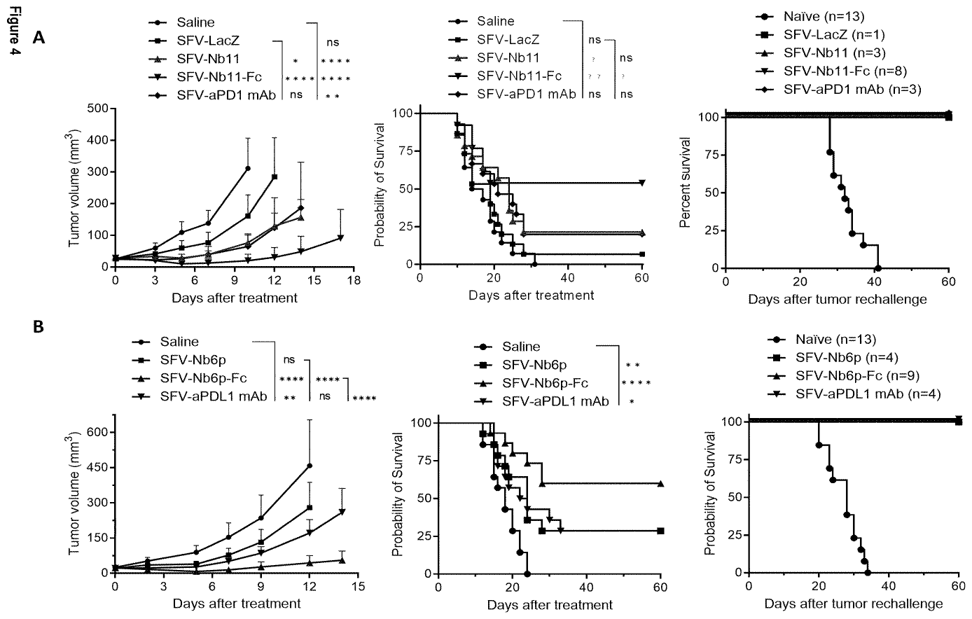

In another embodiment, the immunoconjugate comprises, in addition to a nanobody as defined above, an antibody or antibody fragment, such as an antibody Fc fragment or antibody scFv fragment. In a particular embodiment, the nanobody conjugate comprises an immunoglobulin Fc domain. The “Fc domain” or “fragment crystallizable domain” is the tail region of an antibody that interacts with cell surface receptors called Fc receptors and some proteins of the complement system. In a more particular embodiment, the Fc domain is selected from the group consisting of an IgG Fc domain, an IgM Fc domain, an IgA Fc domain and an IgE Fc domain, more particularly, an IgG Fc domain. In a particular embodiment, the nanobody conjugate comprises two nanobodies bound to an immunoglobulin Fc domain (Fc-dimer). The nanobodies in said Fc-dimer may be the same (Fc-homodimer) or different (Fc-heterodimer). In more particular embodiments the nanobody conjugate is an Fc-homodimer. In one particular embodiment, the Fc domain is a murine IgG domain. In another particular embodiment, the Fc domain is a human IgG domain. In another particular embodiment, the Fc domain is a humanized IgG domain. As shown in the examples below, Fc-dimers comprising anti-PD-L1 and anti-PD1 nanobodies reduce the PD-1/PD-L1 half maximal inhibitory concentration (IC50) by 8- and 40-fold, respectively, and moreover generated very potent antitumor responses in the mouse colon adenocarcinoma MC38 model, resulting in >50% complete regressions and an improved therapeutic efficacy compared to conventional mAbs. These effects were also observed in the mouse B16-OVA melanoma model. Treated animals that had complete remissions remained tumor-free after being rechallenged with MC38 cells (FIG. 4, right panels), suggesting that these treatments were able to generate an efficient memory immune response. An illustrative schematic representation of Fc-dimers may be seen in FIG. 2A.

In one particular embodiment of the first aspect, the nanobody conjugate comprises or consists of a sequence having at least 90%, at least 91%, at least 92%, at least 93%, at least 94%, or at least 95% sequence identity with SEQ ID NO: 9. In a more particular embodiment, the nanobody conjugate comprises or consists of a sequence having at least 96%, at least 97%, at least 98%, or at least 99% sequence identity with SEQ ID NO: 9. In an even more particular embodiment, the nanobody conjugate comprises or consists of a sequence as set forth in SEQ ID NO:9.

In one particular embodiment of the first aspect, the nanobody conjugate comprises or consists of a sequence having at least 90%, at least 91%, at least 92%, at least 93%, at least 94%, or at least 95% sequence identity with SEQ ID NO: 10. In a more particular embodiment, the nanobody conjugate comprises or consists of a sequence having at least 96%, at least 97%, at least 98%, or at least 99% sequence identity with SEQ ID NO:

10. In an even more particular embodiment, the nanobody conjugate comprises or consists of a sequence as set forth in SEQ ID NO:10.

In another embodiment, the nanobody conjugate is a Chimeric Antigen Receptor (CAR). In a particular embodiment, said CAR is encoded in a vector as defined above. Said expression vectors comprising CAR, wherein said CAR comprises a nanobody as described above may be useful to prepare CAR T cells (Chimeric antigen receptor T cells). As known in the art, CAR T cells are T cells that have been genetically engineered to produce an artificial T cell receptor. The present disclosure thus contemplates CAR T cells, wherein the CAR comprises a nanobody as described above. CAR T cells comprising more than one identical or different nanobodies are also contemplated.

In another embodiment, the nanobody conjugate according to the second aspect comprises a nanobody and an effector molecule. “Effector molecule” as used herein, refers to a molecule that selectively binds to a protein and regulates its biological activity.

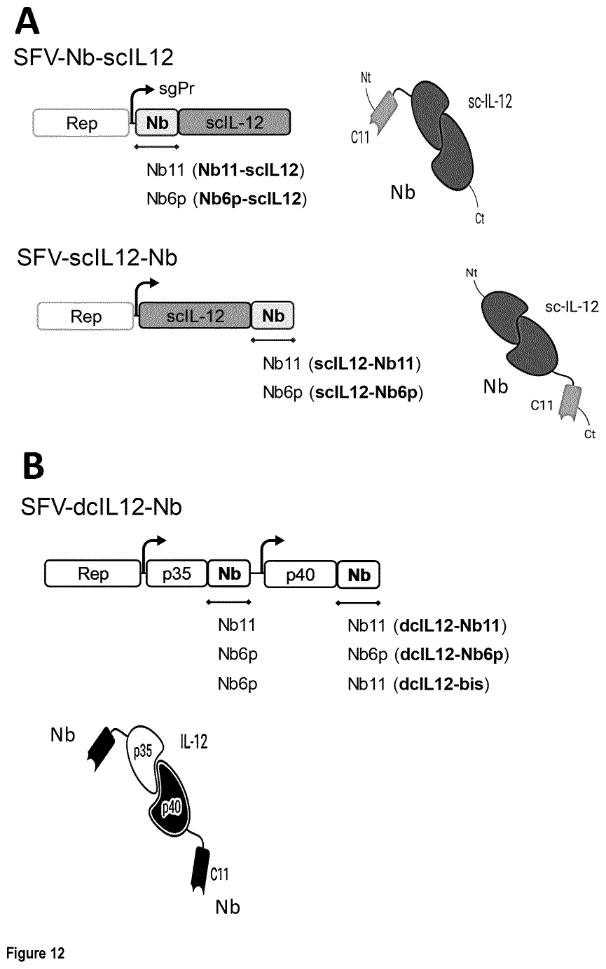

In a particular embodiment, the nanobody conjugate comprises a cytokine. These nanobody conjugates are herein also termed “immunocytokines”, or “ICKs”. The cytokine may be selected from the group consisting of IL-12, IL-2, IL-15, IL-18, IL-21, IL-33, IL-7, IFN-gamma, IFN-alpha, and IFN-beta. Particularly desirable cytokines are those having anti-tumor activity, for example, IL-12. The nanobody may be fused at the amino or carboxy-terminal end of the cytokine, or to both amino and carboxy-terminal ends of the cytokine, and may be fused directly or through a linker. In particular embodiments, the immunocytokine comprises a single-chain version of IL-12. In another particular embodiment, the immunocytokine comprises a double-chain version of IL-12. Illustrative schematic representations of immunocytokines according to the disclosure may be seen in FIG. 12. The advantages of these immunocytokines are related to (i) Synergistic effects on the action of the cytokine and antiPD-1 or antiPD-L1 nanobodies, (ii) a better activity of the cytokine in the tumor due to the fact that the high levels of expression of PD-L1 and PD-1 in the tumors would cause the ICK to be retained in the tumor through the nanobodies, (iii) a reduction of the possible toxicity of the cytokine, as it is retained in the tumor tissue, and (iv) an increase of stability of the nanobodies by fusion to the cytokine.

In some particular embodiments of the second aspect, the nanobody conjugate comprises or consists of a sequence having at least 90%, at least 91%, at least 92%, at least 93%, at least 94%, at least 95%, more particularly, at least 96%, at least 97%, at least 98%, or at least 99% sequence identity with a sequence selected from SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

In a particular embodiment, the nanobody conjugate comprises or consists of a sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18. In another very particular embodiment the nanobody conjugate is a dimer comprising or consisting of SEQ ID NO: 15 and SEQ ID NO: 16. In another very particular embodiment, the nanobody conjugate is a dimer comprising or consisting of SEQ ID NO: 15 and SEQ ID NO: 18. In another very particular embodiment, the nanobody conjugate is a dimer comprising or consisting of SEQ ID NO: 17 and SEQ ID NO: 16. In another very particular embodiment, the nanobody conjugate is a dimer comprising or consisting of SEQ ID NO: 17 and SEQ ID NO: 18.

In another particular embodiment, the nanobody conjugate comprises a polymer. Non-limiting polymers contemplated in this embodiment are those that increases the half-life of the polypeptide, such as polyethylene glycol.

In another particular embodiment, the nanobody conjugate comprises a purification tag, for example, a His-tag.

In another particular embodiment, the nanobody conjugate comprises a detectable marker, for example, a radioactive molecule, fluorescent molecule, chromogenic molecule, or an enzyme. In another embodiment, the nanobody conjugate comprises a nanoparticle, such as gold nanoparticles, quantum dots, or magnetic nanoparticles. In another embodiment, the nanobody conjugate comprises a liposome or vesicle. In another embodiment, the nanobody conjugate comprises a drug, such as a chemotherapeutic agent.

### Polynucleotides, Vectors, Viral Particles and Recombinant Technology

The nanobodies and nanobody conjugates disclosed herein may be administered directly, i.e. in protein form, or as polynucleotides encoding for said polypeptides, wherein the polypeptides are expressed in vitro or in vivo by recipient cells. This means that the polynucleotides are delivered into the cells, for instance, by intratumoral administration of DNA or RNA vectors encoding for the nanobodies or nanobody conjugates, whereby the host cells produce nanobodies or nanobody conjugates in situ.

The third aspect thus refers to a polynucleotide encoding for a nanobody or nanobody conjugate as defined above. All embodiments defined above for the nanobodies or nanobody conjugates also apply to the third aspect. The term “polynucleotide encoding for a nanobody or nanobody conjugate” includes a polynucleotide that encodes for said nanobody or nanobody conjugate and may also contain additional coding and/or non-coding sequences. Polynucleotides of the third aspect may be in the form of DNA or RNA. DNA forms include cDNA, genomic DNA, or synthetic DNA. DNA can be single-stranded or double-stranded. DNA can be a coding strand or a non-coding strand.

In one embodiment of the third aspect, the polynucleotide comprises a sequence encoding for CDR1 as set forth by SEQ ID NO: 1, a sequence encoding for CDR2 as set forth by SEQ ID NO: 2 and a sequence encoding for CDR3 as set forth by SEQ ID NO: 3.

In another embodiment of the third aspect, the polynucleotide comprises a sequence encoding for CDR1 as set forth by SEQ ID NO: 5, a sequence encoding for CDR2 as set forth by SEQ ID NO: 6 and a sequence encoding for CDR3 as set forth by SEQ ID NO: 7.

In one particular embodiment of the third aspect, the polynucleotide comprises or consists of a sequence having at least 90%, at least 91%, at least 92%, at least 93%, at least 94%, or at least 95%, more particularly, at least 96%, at least 97%, at least 98%, or at least 99% sequence identity with SEQ ID NO: 19. In an even more particular embodiment, the polynucleotide has a sequence as set forth in SEQ ID NO:19.

In one particular embodiment of the third aspect, the polynucleotide comprises or consists of a sequence having at least 90%, at least 91%, at least 92%, at least 93%, at least 94%, or at least 95%, more particularly, at least 96%, at least 97%, at least 98%, or at least 99% sequence identity with SEQ ID NO: 20. In an even more particular embodiment, the polynucleotide has a sequence as set forth in SEQ ID NO: 20.

The full-length nucleotide sequence encoding for the nanobody or nanobody conjugate of the present disclosure can generally be obtained by a PCR amplification method or/and a recombination method. Once the polynucleotide sequence has been obtained, the concerned sequences can be obtained in large scale using recombinant methods. Usually, sequences can be obtained by cloning it into a vector, transferring it into cells, and then isolating the sequences from the proliferated host cells by conventional methods. The polynucleotide sequence encoding for the nanobody or nanobody conjugate of the present disclosure can also be obtained by chemical synthesis.

The DNA sequence then can be introduced into various existing DNA molecules (e.g. vectors) and cells known in the art. The invention therefore also relates, in a fourth aspect, to vectors comprising the above-mentioned polynucleotides of the third aspect. In particular embodiments of this fourth aspect, the vector is an expression vector also containing a suitable promoter and, optionally, control sequences. These vectors can be used to express the nanobody or nanobody conjugate in vitro or in vivo.

Non-limitative appropriate expression vectors in the sense of the present disclosure may be derived from virus, such as alphavirus, adenovirus, herpes virus, lentivirus, retrovirus, poxvirus, and Newcastle disease virus. In one embodiment, the expression vector is derived from an alphavirus, for example from Sindbis virus, Venezuelan equine encephalitis virus (VEEV) or Semliki Forest virus (SFV). In a particular embodiment the expression vector is a SFV vector. In a more particular embodiment, the expression vector is a replication-defective alphavirus vector based on Semliki Forest virus (SFV). In a more particular embodiment, the expression vector is based on a propagative SFV vector expressing the viral structural proteins. In an even more particular embodiment, the expression vector is based on a non-propagative SFV vector in which the viral structural proteins have been partially or completely eliminated. In some embodiments, the expression vector comprises the polynucleotide of interest according to the third aspect situated under the control of the subgenomic promoter (sgPr). In particular embodiments, the polynucleotide of interest is situated under the control of the subgenomic promoter (sgPr) fused to a SFV capsid translation enhancer via the 2A self-protease of foot and mouth disease virus. As already mentioned, SFV vectors have shown to successfully deliver the nanobodies and nanobody conjugates as defined above. As shown in the examples below, delivery of the nanobodies described herein through a SFV vector not only enabled a high and local intratumoral nanobody or nanobody conjugate expression but also enhanced their intratumoral activity.

In some particular embodiments of the fourth aspect, the vector comprises or consists of a sequence having at least 90%, at least 91%, at least 92%, at least 93%, at least 94%, or at least 95%, more particularly, at least 96%, at least 97%, at least 98%, or at least 99% sequence identity with a sequence selected from SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and SEQ ID NO: 31. In more particular embodiments, the vector comprises or consists of a sequence selected from SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and SEQ ID NO: 31.

The vectors according to the fourth aspect can be used to produce SFV viral particles which can efficiently infect tumor cells and express the encoded nanobody or nanobody conjugate in vivo. Viral particles are small particles that contains certain proteins from the outer coat of a virus but contain little or no genetic material from the virus and cannot cause an infection. Thus, the present disclosure also contemplates viral particles comprising a vector according to the fourth aspect and a method to produce the same. To produce viral particles a vector comprising polynucleotide encoding for the nanobody or nanobody conjugate is delivered to a host cell together with polynucleotides encoding for certain viral proteins, such as viral capsid and envelope proteins. The cells are then cultured under condition to produce the viral particles, which are then collected and purified by conventional means.

In one embodiment, the method for producing viral particles comprises:
(i) transcribing a vector comprising a polynucleotide encoding for the nanobody or nanobody conjugate; (ii) delivering the RNA to a host cell;
(iii) delivering helper RNAs encoding for capsid and envelope proteins to the host cell;
(iv) culturing the host cell under conditions appropriate to produce viral particles;
(v) harvesting the culture supernatant; and
(vi) collecting the viral particles from the supernatant

In a particular embodiment, the vector comprising a polynucleotide encoding for the nanobody or nanobody conjugate is a SFV vector as defined above. In another particular embodiments the helper RNAs are SFV-helper-C-S219A and SFV-helper-S2. The transcription may be performed by conventional methods. For example, transcription from viral vectors may be done using SP6 RNA polymerase in the presence of m⁷G(′)ppp(5′) G RNA Cap Structure Analog. Delivery of RNA to host cells may also be done by conventional transforming methods, e.g. by electroporation, microinjection, liposome packaging, calcium phosphate coprecipitation, etc. In particular embodiments the RNA encoding for the nanobody or nanobody conjugate and the helper RNA are co-transformed into de host cell.

In another particular embodiment, the host cell for producing the viral particles is a eukaryotic cell, in particle, a higher eukaryotic cell, more particularly a mammal cell, for example selected from CHO-K1, BHK, Vero, and Vero E6, and even more particularly, a BHK cell. Such as a BHK-21. Depending on the host cell used, the medium used in the culture may be selected from various conventional media. The culture is performed under conditions suitable for the host cells growth. Harvesting the supernatant and collecting of the viral particles contained therein can also be performed by conventional methods. For example, harvesting may be done by centrifugation and the viral particles may be collected by ultracentrifugation.

The present disclosure also refers to the viral particles obtainable or obtained by the methods described above.

As mentioned above, the vectors of the fourth aspect or the host cells of the fifth aspect, comprising polynucleotides encoding for a nanobody or nanobody conjugate as defined above, may also be used to recombinantly produce the nanobody or nanobody conjugate in vitro, which may then be delivered to tumoral cells as a recombinant product. The seventh aspect thus provides a method for producing nanobodies or nanobody conjugates as defined above, said method comprising the steps of: (a) culturing a host cell comprising an expression vector as defined above encoding for a nanobody or nanobody conjugate under conditions suitable for producing the nanobodies or nanobody conjugates, thereby obtaining a culture containing said nanobodies or nanobody conjugates; and (b) isolating or recovering said nanobodies or nanobody conjugates from said culture.

In general, the host cell for expressing the nanobody or nanobody conjugate in vitro can be a prokaryotic cell, such as a bacterial cell; or a lower eukaryotic cell, such as a yeast cell; or a higher eukaryotic cell, such as a mammalian cell. Representative examples are: bacterial cells such as Escherichia coli, Streptomyces or Salmonella typhimurium, fungal cells such as yeast, insect cells, such as Drosophila S2 or Sf9, animal cells, such as BHK, CHO, Vero, Vero E6, COS7, 293 cells, and the like. In one embodiment of the seventh aspect, the host cell is E. coli. In another embodiment, the host cell is selected from Saccharomyces cerevisiae and Pichia pastoris. In another particular embodiment, the host cell is a mammal cell, for example selected from BHK, CHO-K1, Vero, and Vero E6, more particularly a BHK cell, such as BHK-21.

The transformation of the host cell with the recombinant DNA can be performed using conventional techniques well known to those skilled in the art. When the host is a prokaryotic organism such as E. coli, competent cells capable of absorbing DNA can be harvested after the exponential growth phase and treated with the CaCl₂) method. The procedures used are well known in the art. Another method is to use MgCl₂. If necessary, conversion can also be performed by electroporation. When the host is eukaryotic, transfection can be done by electroporation, microinjection, liposome packaging, calcium phosphate coprecipitation, and the like. The obtained transformants can be cultured in a conventional manner to express the polypeptide encoded by the gene of the present invention. As already mentioned above, depending on the host cells used, the medium used in the culture may be selected from various conventional media and the culture is performed under conditions suitable for the host cells growth. After the host cells are grown to an appropriate cell density, the selected promoter may be induced by a suitable method (such as temperature shift or chemical induction) and the cells are incubated for a further period of time.

The recombinant polypeptide in the above method may be expressed intracellularly, or on the cell membrane, or secreted extracellularly. If necessary, the recombinant protein can be isolated and purified by various separation methods by utilizing its physical, chemical and other characteristics. These methods are well-known to those skilled in the art. Examples of these methods include, but are not limited to: conventional renaturation treatment, treatment with a protein precipitation agent (salting out method), centrifugation, osmotic disruption, ultracentrifugation, molecular sieve chromatography (gel filtration), adsorption layer analysis, ion exchange chromatography, high performance liquid chromatography (HPLC), and various other liquid chromatography techniques and the combinations thereof.

The present disclosure also contemplates recombinant nanobodies or nanobody conjugates obtainable or obtained by the above methods.

The nanobody or nanobody conjugate may also be delivered to cells in vivo by a non-viral mode, e.g. by delivering a RNA or DNA vector to the target cells without being vehiculated through viral particles. As shown in the examples below, this last option was also successfully used to deliver a dimeric nanobody as defined above to tumoral cells and resulted in antitumor effects comparable to those obtained with viral particles. An additional advantage of the DNA system is that no neutralizing antibodies against SFV will be induced, allowing for multiple administrations. The nanobody or nanobody conjugate as defined above may also be delivered as RNA or DNA using a plasmid in which the SFV vector sequence is placed under a eukaryotic promoter, e.g. CMV promoter. In this DNA/RNA system, the SFV RNA is transcribed in transfected cells, leading to high transgene expression levels and apoptosis in a similar way to SFV-infected cells

### Compositions

An eight aspect of this disclosure refers to a pharmaceutical composition comprising a nanobody, or a nanobody conjugate, or an expression vector, or a host cell, or a viral particle, all of them as defined above, together with pharmaceutically acceptable excipients and/or carriers. All embodiments described above for the nanobody, nanobody conjugate, expression vector, host cell, or viral particle, also apply to the pharmaceutical composition. The expression “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

The election of the pharmaceutical formulation will depend upon the nature of the active compound and its route of administration. Any route of administration may be used. In some embodiments, the route of administration is parenteral, and the composition is then appropriate for parenteral administration. In a particular embodiment, the route of administration is by injection. In a more particular embodiment, the route of administration is systemic, for example, by intramuscular, intravenous, intraarterial, intraperitoneal, subcutaneous, or transdermal injection. In a particular embodiment, the route of administration is local, for example, intratumoral injection. The intratumoral route of administration is particularly suitable for delivering the nanobody or nanobody conjugate of the invention by a vector, for example, a viral vector or by viral nanoparticles, although also the nanobody or nanobody conjugate of the invention can be administered by this route in protein form. Topical administration is also contemplated, such that the pharmaceutical composition may be a topical composition.

The pharmaceutical compositions may be in any form, including, among others, tablets, pellets, capsules, aqueous or oily solutions, suspensions, emulsions, aerosols, or dry powdered forms suitable for reconstitution with water or other suitable liquid medium before use, for immediate or retarded release.

The appropriate excipients and/or carriers, and their amounts, can readily be determined by those skilled in the art according to the type of formulation being prepared. Examples of suitable pharmaceutically acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The term “carrier” is to be understood as a pharmaceutically acceptable vehicle. The carrier can be organic, inorganic, or both. Suitable carriers well known to those of skill in the art and include, without limitation, large, slowly metabolized macromolecules such as proteins, polysaccharides, polylactic acids, polyglycolic acids, polymeric amino acids, amino acid copolymers, lipid aggregates (such as oil droplets or liposomes) and inactive virus particles. Carriers may also include, saline, buffer, dextrose, water, glycerol, ethanol, and the combinations thereof. In particular embodiments, the carries may be a polycationic polymer, a vesicle, a liposome, or a nanoparticle.

Usually, the pharmaceutical composition comprises a therapeutically effective amount of the nanobody, nanobody conjugate, expression vector, host cell, or viral particle. The expression “therapeutically effective amount” as used herein, refers to the amount of a compound that, when administered, is sufficient to prevent development of, or alleviate to some extent, one or more of the symptoms of the disease which is addressed. The particular dose of compound administered according to this disclosure will of course be determined by the particular circumstances surrounding the case, including the compound administered, the route of administration, the particular condition being treated, and the similar considerations. The therapeutically effective amount may be 0.001-50 wt %, in particular 0.01-10 wt %, and more preferably 0.1-5 wt % of the nanobody, nanobody conjugate, expression vector, host cell, or viral particle. The amount of active ingredient administered (or dose) may be from about 10 micrograms/kilogram body weight to about 50 milligrams/kilogram body weight per day, in particular the dose may be from about 100 micrograms/kilogram body weight to about 40 milligrams/kilogram body weight, more in particular, the dose may be from about 1 milligrams/kilogram body weight to about 30 milligrams/kilogram body weight. In addition, the nanobodies, nanobody conjugates, vectors or viral particles of the present disclosure may also be used with other therapeutic agents.

The disclosure also contemplates diagnostic compositions comprising a nanobody, or a nanobody conjugate, as defined above.

Herein disclosed is also a kit of parts that comprises:
(a) the nanobody, nanobody conjugate, vector, or viral particles as defined above, optionally together with pharmaceutically acceptable excipients or carriers;
(b) optionally a further therapeutic agent; and
(c) optionally, instructions for its use.

Herein disclosed is also a vessel or injection device which comprises the nanobody, nanobody conjugate, vector, or viral particles as defined above, preferably together with pharmaceutically acceptable excipients or carriers.

The disclosure also contemplates a kit of parts that comprises:
(a) the nanobody or nanobody conjugate as defined above;
(b) optionally further means for performing a diagnosis (e.g. buffers, reagents, controls); and
(c) optionally, instructions for its use.

### Uses

The disclosure provides, in the ninth aspect, a pharmaceutical composition, or a nanobody conjugate, or an expression vector, or a host cell, or a viral particle, or a kit of parts, all of them as defined above, for use in therapy. In a particular embodiment, the therapy is immunotherapy. In another embodiment, the therapy comprises blocking, or inhibiting, the binding between human PD-L1 and PD-1. The invention also contemplates the nanobody, nanobody conjugate, expression vector, host cell, viral particle, or pharmaceutical composition for use as immune checkpoint inhibitor.

A tenth aspect provides a nanobody, or a nanobody conjugate, or an expression vector, or a host cell, or a viral particle, or a pharmaceutical composition, or a kit of parts, all of them as defined above, for use in treating cancer. In other words, the tenth aspect provides a pharmaceutical composition, or a nanobody, or a nanobody conjugate, or an expression vector, or a host cell, or a viral particle, or kit of parts, all of them as defined above, for use in treating a tumor. As used herein the terms “cancer” or “tumor” are used interchangeably and refer as generally understood in the art to a malignant abnormal cell growth with the potential to invade or spread to other parts of the body.

“Treating cancer” in the sense of the present disclosure includes a prophylactic treatment before the clinical onset of cancer or a therapeutic treatment after the clinical onset of cancer and may be achieved by arresting the development or reversing the symptoms of cancer.

In a particular embodiment of the tenth aspect, the nanobody, nanobody conjugate, expression vector, host cell, viral particle, kit of parts, or pharmaceutical composition are for use in combination therapy with surgery, radiation or a further therapeutic agent for the treatment of cancer. The nanobody, nanobody conjugate, expression vector, host cell, viral particle, kit of parts, or pharmaceutical composition, and the surgery, radiation, or further therapeutic agent may be administered sequentially, simultaneously or within a therapeutic interval. In a particular embodiment the combination therapy comprises surgery. In another particular embodiment the combination therapy comprises radiation. In a particular embodiment the combination therapy comprises an antitumoral agent. The antitumoral agent may be a cytotoxic agent, such as, for example, alkylating agents, antimetabolites, including folate antagonists, purine and pyrimidine analogs, antibiotics and other natural products, including anthracyclines and vinca alkaloids, and antibodies, which improve specificity. The antitumoral agent may also be a hormonal agent or a signal transduction inhibitor. In a particular embodiment the combination therapy comprises chemotherapy. The combination therapy may also comprise a different immunotherapy. For example, in a particular embodiment the combination may comprise activated natural killer cells, (CAR) T-cells, tumor-infiltrating lymphocytes or tumor antigen-loaded dendritic cells.

The cancer which can be treated according to the tenth aspect is not particularly limited. Non-limiting types of tumors or cancers that can be treated in the sense of the present description are carcinomas, sarcomas, and hematologic tumors. For example, the cancer to be treated may be a carcinoma, such as adenocarcinoma, sarcoma, osteosarcoma, chondrosarcoma, leiomyosarcomas, rhabdomyosarcomas, mesotheliomas, fibrosarcomas, angiosarcomas, liposarcomas, gliomas, myxosarcomas, mesenchymous, a hematologic cancer, such as myeloma, leukemia, or lymphoma, and mixed type tumors, such as adenosquamous carcinomas, mixed mesodermal tumors, carcinosarcomas, or teratocarcinomas. Carcinomas are the predominant cancers and are cancers of epithelial cells or cells covering the external or internal surfaces of organs, glands, or other body structures (e.g., skin, uterus, lung, breast, prostate, stomach, bowel), and which tend to metastasize. Carcinomas may be adenocarcinomas, for example, of the breast, lung, colon, prostate or bladder, and squamous cell carcinomas. Sarcomas are derived from connective or supportive tissue (e.g., bone, cartilage, tendons, ligaments, fat, and muscle). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors are derived from bone marrow and lymphatic tissue. Hematologic tumors may be myelomas, which originate in the plasma cells of bone marrow; leukemias which may be “liquid tumors” and are tumors of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type tumors, such as adenosquamous carcinomas, mixed mesodermal tumors, carcinosarcomas, or teratocarcinomas also exist. All these types of tumors, as well as others that would be apparent to the skilled person, are contemplated in the sense of the present disclosure.

Tumors that may be treated in the sense of the present disclosure may also be named based on the organ in which they originate i.e., the “primary site,” for example, tumor, or cancer, of the breast, brain, lung, liver, skin, prostate, testicle, bladder, colon and rectum, cervix, uterus, blood, lymph, etc. This naming often persists even if the tumor metastasizes to another part of the body that is different from the primary site. In this sense, the cancer contemplated in the tenth aspect may be, but is not limited to, a cancer of the gastrointestinal tract, respiratory system, nervous system, hematopoietic system, epitelium, vascular system, reproductive system (such as cervical, ovarian, uterine, vaginal, and vulvar), urinary tract, endocrine system, skin, heart, brain, eyes, testes, muscles, bones or breasts.

In particular embodiments the cancer to be treated is selected from colon adenocarcinoma and melanoma.

The eleventh to thirteenth aspects of the present disclosure are related to use of the nanobody, nanobody conjugate or kit of parts as defined above in analysis (based in detecting human PD-L1 and/or PD-1 molecules) or diagnosis. In most embodiments of these aspects, the nanobody or nanobody conjugate is linked to a detectable molecule.

All the embodiments described above for the nanobody, nanobody conjugate, expression vector, host cell, viral particle, pharmaceutical composition, or kit of parts also apply to the therapeutic uses described in this section.

For completeness, the present description is also disclosed in the following numbered embodiments:
1. A single domain antibody (VHH or nanobody) comprising the following complementary determining regions (CDRs):
(i) CDR1 as set forth by SEQ ID NO: 1, CDR2 as set forth by SEQ ID NO: 2 and CDR3 as set forth by SEQ ID NO: 3.
2. The nanobody according to embodiment 1, which
(i) is an anti-PD-L1 nanobody comprising CDR1 as set forth by SEQ ID NO: 1, CDR2 as set forth by SEQ ID NO: 2 and CDR3 as set forth by SEQ ID NO: 3, and is able to inhibit binding between PD-L1 and PD-1.
3. The nanobody according to any one of embodiments 1-2, which blocks the binding between PD-L1 and PD-1 with half maximal inhibitory concentration (IC50) of 6 nM or lower.
4. The nanobody according to any one of embodiments 1-3, which inhibits the interaction of PD-1 and PD-L1 for human and mouse orthologs.
5. The nanobody according to any one of embodiments 1-4, comprising or consisting of:
(i) a sequence as set forth in SEQ ID NO:4, or a sequence having at least 90%, or at least 95%, or at least 98%, or at least 99%, or 100% identity with SEQ ID NO: 4.
6. The nanobody according to the preceding embodiment, comprising or consisting of a conservative variant of SEQ ID NO: 4.
7. A nanobody conjugate comprising a nanobody according to any one of embodiments 1-6.
8. The nanobody conjugate according to the preceding embodiment, comprising two, three, four, five or six nanobodies.
9. The nanobody conjugate according to the preceding embodiment, that is monospecific.
10. The nanobody conjugate according to embodiment 8, that is at least bispecific.
11. The nanobody conjugate according to any one of embodiments 7-10, comprising at least one nanobody according to any one of embodiments 1-6 and an effector molecule.
12. The nanobody conjugate according to any one of embodiments 7-11, comprising two nanobodies as defined in any one of embodiments 1-6 fused to an immunoglobulin Fc domain.
13. The nanobody conjugate according to the preceding embodiment, comprising or consisting of SEQ ID NO: 9, or a sequence having at least 90%, at least 95%, at least 96%, at least 96%, at least 98%, or at least 99% sequence identity with SEQ ID NO: 9.
14. The nanobody conjugate according to any one of embodiments 7-11, comprising at least one nanobody according to any one of embodiments 1-6 and a cytokine.
15. The nanobody conjugate according to the preceding embodiment, wherein the cytokine is selected from the group consisting of IL-12, IL-2, IL-15, IL-18, IL-21, IL-33, IL-7, IFN-gamma, IFN-alpha, and IFN-beta.
16. The nanobody conjugate according to the preceding embodiment, wherein the cytokine is IL-12.
17. The nanobody conjugate according to the preceding embodiment, comprising or consisting of a sequence selected from the group consisting of SEQ ID NO: 11, SEQ ID NO: 12, SEQ ID NO: 15, and SEQ ID NO: 16 or a sequence having at least 90%, at least 95%, at least 96%, at least 96%, at least 98%, or at least 99% sequence identity with a sequence selected from the group consisting of SEQ ID NO: 11, SEQ ID NO: 12, SEQ ID NO: 15, and SEQ ID NO: 16
18. The nanobody conjugate according to the preceding embodiment, which is a dimer comprising or consisting of SEQ ID NO: 15 and SEQ ID NO: 16, a dimer comprising or consisting of SEQ ID NO: 15 and SEQ ID NO: 18, or a dimer comprising or consisting of SEQ ID NO: 17 and SEQ ID NO: 16.
19. The nanobody conjugate according to any one of embodiments 7-18, which blocks the binding between PD-L1 and PD-1 with half maximal inhibitory concentration (IC50) of 3 nM or lower.
20. The nanobody conjugate according to the preceding embodiment, which blocks the binding between PD-L1 and PD-1 with half maximal inhibitory concentration (IC50) of 1 nM or lower, in particular of 0.5 nM or lower, more in particular of 0.1 nM or lower.
21. A single domain antibody (VHH or nanobody) comprising the following complementary determining regions (CDRs): CDR1 as set forth by SEQ ID NO: 5, CDR2 as set forth by SEQ ID NO: 6 and CDR3 as set forth by SEQ ID NO: 7.
22. The nanobody according to embodiment 21, which is an anti-PD-1 nanobody comprising CDR1 as set forth by SEQ ID NO: 5, CDR2 as set forth by SEQ ID NO: 6 and CDR3 as set forth by SEQ ID NO: 7, and is able to inhibit binding between PD-L1 and PD-1.
23. The nanobody according to any one of embodiments 21-22, which blocks the binding between PD-L1 and PD-1 with half maximal inhibitory concentration (IC50) of 6 nM or lower.
24. The nanobody according to any one of embodiments 21-23, which inhibits the interaction of PD-1 and PD-L1 for human and mouse orthologs.
25. The nanobody according to any one of embodiments 21-24, comprising or consisting of:
a sequence as set forth in SEQ ID NO:8, or a sequence having at least 90%, or at least 95%, or at least 98%, or at least 99% identity with SEQ ID NO: 8.
26. The nanobody according to the preceding embodiment, comprising or consisting of a conservative variant of SEQ ID NO: 8.
27. A nanobody conjugate comprising a nanobody according to any one of embodiments 21-26.
28. The nanobody conjugate according to the preceding embodiment, comprising two, three, four, five, or six nanobodies.
29. The nanobody conjugate according to the preceding embodiment, that is monospecific.
30. The nanobody conjugate according to embodiment 28, that is at least bispecific.
31. The nanobody conjugate according to any one of embodiments 27-30, comprising at least one nanobody according to any one of embodiments 20-26 and an effector molecule.
32. The nanobody conjugate according to any one of embodiments 27-31, comprising two nanobodies as defined in any one of embodiments 21-26 fused to an immunoglobulin Fc domain.
33. The nanobody conjugate according to the preceding embodiment, comprising or consisting of SEQ ID NO: 10, or a sequence having at least 90%, at least 95%, at least 96%, at least 97%, at least 98%, or at least 99% sequence identity with SEQ ID NO: 10.
34. The nanobody conjugate according to any one of embodiments 27-31, comprising at least one nanobody according to any one of embodiments 21-26 and a cytokine.
35. The nanobody conjugate according to the preceding embodiment, wherein the cytokine is selected from the group consisting of IL-12, IL-2, IL-15, IL-18, IL-21, IL-33, IL-7, IFN-gamma, IFN-alpha, and IFN-beta.
36. The nanobody conjugate according to the preceding embodiment, wherein the cytokine is IL-12.
37. The nanobody conjugate according to the preceding embodiment, comprising or consisting of a sequence selected from the group consisting of SEQ ID NO: 13, SEQ ID NO: 14, SEQ ID NO: 17, and SEQ ID NO: 18, or a sequence having at least 90%, at least 95%, at least 96%, at least 97%, at least 98%, or at least 99% sequence identity with a sequence selected from the group consisting of SEQ ID NO: 13, SEQ ID NO: 14, SEQ ID NO: 17, and SEQ ID NO: 18.
38. The nanobody conjugate according to the preceding embodiment, which is a dimer comprising or consisting of, SEQ ID NO: 17 and SEQ ID NO: 18, a dimer comprising or consisting of SEQ ID NO: 15 and SEQ ID NO: 18, or a dimer comprising or consisting of SEQ ID NO: 17 and SEQ ID NO: 16.
39. The nanobody conjugate according to any one of embodiments 27-38, which blocks the binding between PD-L1 and PD-1 with half maximal inhibitory concentration (IC50) of 3 nM or lower.
40. The nanobody conjugate according to the preceding embodiment, which blocks the binding between PD-L1 and PD-1 with half maximal inhibitory concentration (IC50) of 1 nM or lower, in particular of 0.5 nM or lower, more in particular of 0.1 nM or lower.
41. The nanobody according to any one of embodiments 1-6 or 21-26, or the nanobody conjugate according to any one of embodiments 7-20 or 27-40, that is humanized.
42. A polynucleotide encoding for a nanobody as defined in anyone of embodiments 1-6 or 21-26, or for a nanobody conjugate as defined in any one of embodiments 7-20 or 27-40.
43. A polynucleotide according to the preceding embodiment comprising or consisting of SEQ ID NO: 19 or a sequence at least 95%, at least 96%, at least 97%, at least 98%, or at least 99% sequence identity with SEQ ID NO: 19.
44. A polynucleotide according to the preceding embodiment comprising or consisting of SEQ ID NO: 20 or a sequence at least 95%, at least 96%, at least 97%, at least 98%, or at least 99% sequence identity with SEQ ID NO: 20.
45. An expression vector comprising the polynucleotide as defined in any one of embodiments 42-44.
46. The expression vector according to the preceding embodiment, wherein the expression is derived from an alphavirus or an adenovirus.
47. The expression vector according to the preceding embodiment, wherein the vector is derived from an alphavirus selected from Sindbis virus, Venezuelan equine encephalitis virus (VEEV), or Semliki Forest virus (SFV).
48. The expression vector according to the preceding embodiment, wherein the vector is derived from SFV, in particular, the vector is a replication-defective alphavirus vector based on SFV.
49. The expression vector according to the preceding embodiment, wherein the vector comprises or consists on a sequence selected from the group consisting of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and SEQ ID NO: 31, or a sequence having at least 90%, at least 95%, at least 96%, at least 97%, at least 98%, or at least 99% sequence identity with any of these sequences.
50. A host cell comprising a polynucleotide as defined in any one of embodiments 42-44, or an expression vector as defined in any one of embodiments 45-49.
51. The host cell according to the preceding embodiments, wherein the cell is selected from the group consisting of a CHO-K1, BHK, Vero, and Vero E6 cell, in particular, a BHK cell.
52. A viral particle comprising an expression vector according to any one of embodiments 45-49.
53. The viral particle according to embodiment 52, further comprising SFV capsid and envelope proteins.
54. A pharmaceutical composition comprising a nanobody as defined in any one of embodiments 1-6 or 21-26, or a nanobody conjugate as defined in any one of embodiments 7-20 or 27-40, or an expression vector as defined in any one of embodiments 45-49, a host cell as defined in any one of embodiments 50-51, or a viral particle as defined in any one of embodiments 52-53, together with pharmaceutically acceptable excipients and/or carriers.
55. A nanobody as defined in any one of embodiments 1-6 or 21-26, or a nanobody conjugate as defined in any one of embodiments 7-20 or 27-40, or an expression vector as defined in any one of embodiments 45-49, a host cell as defined in any one of embodiments 50-51, or a viral particle as defined in any one of embodiments 52-53, or a pharmaceutical composition as defined in embodiment 54, for use in therapy.
56. A nanobody as defined in any one of embodiments 1-6 or 21-26, or a nanobody conjugate as defined in any one of embodiments 7-20 or 27-40, or an expression vector as defined in any one of embodiments 45-49, a host cell as defined in any one of embodiments 50-51, or a viral particle as defined in any one of embodiments 52-53, or a pharmaceutical composition as defined in embodiment 54, for the prevention and/or treatment of cancer.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1. Local Delivery of Nanobodies Targeting the PD-1/PD-L1 Axis with a Self-Amplifying RNA Viral Vector Induces Potent Antitumor Responses

### Methods

### Cell Lines and Animals

BHK-21 cells (ATCC-CCL 10) were cultured in GMEM-BHK21 (Thermo Fisher, Waltham, MA) supplemented with 5% fetal bovine serum (FBS), 10% tryptose phosphate broth, 2 mM glutamine, 20 mM HEPES and antibiotics (100 μg/mL streptomycin and 100 U/mL penicillin) (complete GMEM). HEK-293 (ATCC-CRL-3216) were cultured in DMEM (Gibco, BRL, UK) supplemented with 10% FBS, 2 mM glutamine and antibiotics. MC38 cells, a kind gift from Dr. Karl E. Hellström (University of Washington, Seattle, WA), were cultured in RPMI-1640 medium (Lonza, Switzerland) supplemented with 10% FBS, 2 mM glutamine, 20 mM HEPES, antibiotics and 50 UM 2-mercaptoethanol. B16-OVA cells were kindly provided by Dr. Lieping Chen and cultured in RPMI-1640 medium supplemented with 10% FBS, 2 mM glutamine, 20 mM HEPES, antibiotics and 400 μg/mL of Geneticin. Four or six-week-old female C57BL/6 mice were purchased from Envigo (Barcelona, Spain). Animal studies were approved by the Universidad de Navarra ethical committee (study number 078-19) for animal experimentation under Spanish regulations.

One adult female llama (Lama glama) from Montevideo municipal zoo (Uruguay) was used for immunization and construction of single-domain antibody library. The protocol was approved by the Parque Lecocq ethical committee and manipulation of the llama was performed by veterinarians.

### In Vitro PD-1/PD-L1 Inhibition Assays

The ability of nanobodies (Nbs) to inhibit the binding of PD-1 with PD-L1 was evaluated by competitive ELISA. For human molecules, plates were coated with 0.5 μg/mL hPD-1-Fc (R&D, Minneapolis, MN) in PBS o/n at 4° C. After blocking with PBS-0.5% BSA, biotinylated hPD-L1 fused to human IgG Fc (BPS Bioscience, San Diego, CA) was added at 0.25 μg/mL, diluted in PBS-0.2% BSA, together with different concentrations of anti-PD-1 or anti-PD-L1 Nbs (or mAbs). Detection of hPD-L1 bound to hPD-1 was performed using streptavidin conjugated to peroxidase. For mouse molecules, a similar protocol was followed. In this case, 1 μg/mL mPD-L1-Fc (BioLegend, San Diego, CA) in PBS was used to coat the plates, and biotinylated mPD-1-Fc (BPS Bioscience) was used at 0.25 μg/mL. Commercial antibodies were included in these assays as controls of PD-1/PD-L1 inhibition: anti-mouse PD-1 (clone RMPI-14, BioXCell, Lebanon, NH), anti-mouse PD-L1 (clone 10F.9G2, BioXCell), nivolumab (Bristol Myers Squibb, New York, NY) and atezolizumab (Roche, Basel, Switzerland). The signal of wells incubated without Nbs or antibodies were considered 100% of PD-1/PD-L1 binding.

### Generation of SFV Vectors Encoding Nbs

The sequences of Nb11 (anti-PD-1) and Nb6p (anti-PD-L1) Nbs were cloned into the SFVb12A plasmid [Rodriguez-Madoz J R, et al, Mol Ther. 2005] using Apa I restriction sites, generating SFV-Nb11 and SFV-Nb6p vectors, respectively. In these vectors, a hemagglutinin (HA) tag was included at the carboxy-terminus of each Nb sequences to allow protein detection. The sequences of Nbs fused to mouse Fc domains (including the hinge region) were synthetized by Genscript (Piscataway, NJ) and subcloned into the SFVb12A vector using Apa I restriction sites, generating SFV-Nb11-Fc and SFV-Nb6p-Fc vectors, respectively. Nb11 was fused to mIgG1 Fc (Nb11-Fc) and Nb6p to mIgG2a Fc (Nb6p-Fc). To generate SFV-aPD1, the sequence of an anti-PD-1 mouse monoclonal antibody (mAb) [US 2010/0028330] was synthetically requested from GenScript. This synthetic gene, which contains the sequences corresponding to the heavy (IgG1 isotype) and light (lambda) chains of the aPD-1 mAb fused by the autoprotease 2A sequence from foot and mouth disease virus preceded by a furin cleavage sequence, was subcloned into the Apa I site of SFVb12A following the same strategy previously used to generate SFV-aPDL1, which codes for a mAb against mouse PD-L1 with the same structure [Ballesteros-Briones M C, et al, Mol Ther. 2019]. SFV-LacZ had been previously described [Quetglas J I, et al, Gene Ther. 2012].

For the non-viral delivery experiment, SFV-Nb11-Fc and SFV-LacZ replicons were subcloned into pBK-T-SFV plasmid [Berglund P, et al. Nat Biotechnol. 1998] using Apa I restriction sites.

### Production of Recombinant SFV Viral Particles

SFV RNA was transcribed in vitro from SFV plasmids using SP6 RNA polymerase (Promega) and m⁷G(′)ppp(5′) G RNA Cap Structure Analog (New England Biolabs, Ipswich, MA). RNA synthesis and delivery into BHK-21 cells by electroporation was performed as described previously [Liljeström P, et al. Curr Protoc Mol Biol. 2001]. To produce SFV viral particles, two helper RNAs (SFV-helper-C-S219A and SFV-helper-S2) were co-electroporated together with the recombinant RNA, providing the SFV capsid and envelope proteins in trans, respectively [Smerdou C, et al. J Virol. 1999]. Forty eight hours after electroporation, supernatants were harvested, and Viral particles were purified by ultracentrifugation as described [Ballesteros-Briones M C, et al, Mol Ther. 2019]. For titration of Viral particles, BHK-21 cells were infected with serial dilutions of the SFV vectors. For SFV-Nb11 and SFV-Nb6p, indirect immunofluorescence was performed using a mouse anti-HA primary antibody (BioLegend) and a secondary anti-mouse IgG antibody conjugated to Alexa-488 (Invitrogen, Waltham, MA). For antibodies with Fc domains, a direct immunofluorescence was performed using the same anti-mouse IgG antibody. For SFV-LacZ, infected cells were treated with X-Gal staining solution and blue cells were counted as positive. Vector titers ranged from 2-6×10¹⁰ Viral particles/mL for SFV vectors coding for Nbs or antibodies, and 0.5-2×10¹¹ Viral particles/mL for SFV-LacZ.

Expression of Nbs from SFV Vectors In Vitro

Expression of Nbs in vitro was evaluated using BHK-21 cells infected with SFV Viral particles. 10⁶ BHK-21 cells were seeded in six-well plates and after 24 h they were infected with SFV Viral particles at a multiplicity of infection (MOI) of 20. Viral particles were diluted in 300 μL of MEM supplemented with 2 mM glutamine and 0.2% BSA, cells were infected for 1 h at 37° C. after which infection medium was replaced with complete GMEM. To evaluate expression of Nb11-Fc from pBK-T-SFV plasmid, 5×10⁵ BHK-21 cells/well in 6-well plates were transfected with 2 μg of plasmid/well using lipofectamine-2000 (Thermo Fisher).

Supernatants and cell extracts from infected or transfected cells were collected after 24 h to evaluate antibody expression. Supernatants were centrifuged at 10,000 g for 5 min at 4° C. to eliminate cell debris. Cells were washed three times with PBS before incubating them with cold lysis buffer (50 mM Tris-HCl PH 7.5, 1% NP-40, 150 mM NaCl, 2 mM EDTA, and Protease Inhibitor Cocktail, Roche) for 10 min at 4° C. Lysed cells were centrifuged at 6,000 g 10 min at 4° C., and supernatants (containing proteins from lysed cells) were collected for analysis. Samples were stored at −80° C. until use.

### Antibody Quantification by ELISA

To quantify monomeric Nbs in samples from in vitro and in vivo experiments, specific hPD-1 or mouse PD-L1 (mPD-L1) binding ELISAs were performed. Ninety-six-well ELISA plates were coated with hPD-1 fused to human IgG1 Fc (R&D) at 0.2 μg/mL, or with mPD-L1 fused to human IgG1 Fc (BioLegend) at 1 μg/mL in PBS o/n at 4° C. Next day, plates were blocked with 0.5% BSA in PBS for 1 h at RT, washed with PBST, and incubated with samples diluted in PBST-0.2% BSA for 2 h at RT. For detection, an anti-HA antibody (BioLegend) was used, followed by incubation with a polyclonal antibody against mouse IgG conjugated to peroxidase (Sigma, St. Louis, MO). Standard curves were included in every assay, using the corresponding purified Nb produced in E. coli, as described previously [Silva-Pilipich N, et al Biomedicines. 2020]. To compare expression of Nb-Fc constructs and conventional mAbs from in vitro experiments, samples were analyzed by a sandwich ELISA for detection of total mouse IgG. ELISA plates were coated with a polyclonal anti-mouse IgG (Abcam, UK) in PBS o/n at 4° C., blocked with 0.5% BSA in PBS 1 h at RT, and samples diluted in PBST-0.2% BSA were incubated for 2 h at RT. Antibodies against mIgG1 or mIgG2a conjugated to peroxidase were used for detection (Abcam, UK). The assays were developed using tetramethylbenzidine (TMB) substrate and stopped with H₂SO₄ 2 N. Absorbance was read at 450 nm on a Fluostar Optima Reader. Standard curves were included in every assay, using purified Nb11-Fc or Nb6p-Fc from the supernatants of BHK-21 cells electroporated with SFV-Nb11-Fc and SFV-Nb6p-Fc RNAs. Nb6p-Fc was purified using a protein A Sepharose column and the ÄKTA purification system (Cytiva, Marlborough, MA), following manufacturer's instructions. Nb11-Fc was concentrated using a 50 kDa Amicon filter (Millipore, Burlington, MA). Quantification of Nbs was done by Coomassie blue staining using BSA as standard.

### Western Blot Analysis of Antibodies

Samples from BHK-21 cells infected with SFV vectors were analyzed by Western blot under reducing (with dithiothreitol, DTT) or non-reducing conditions (without DTT), in 10% or 8% polyacrylamide gels respectively, for both Fc-fused Nbs and mAbs. For monomeric Nbs, 15% polyacrylamide gels were used due to their smaller size. Detection of monomeric Nbs or Fc-fused Nbs was performed using the same antibodies described for the ELISA assays, diluted in TBS-0.05% Tween 20-5% non-fat milk. Membranes were developed with Lumigen ECL Ultra substrate (Beckman Coulter, Brea, CA) and images acquired in ChemiDoc Imaging System (BioRad, Hercules, CA).

### Tumor Induction and Treatment

C57BL/6J mice were subcutaneously (s.c.) injected in the right flank with 5×10⁵ tumor cells diluted in saline solution. Seven-ten days after tumor inoculation, SFV vectors were administered intratumorally (i.t.) in a total volume of 50 μL, diluted in saline solution. One dose of 3×10⁸ Viral particles/tumor was used in all the experiments except in the bilateral tumor model. Control untreated mice received the same volume of saline solution. For bilateral tumor experiment, 5×10⁵ and 3×10⁵ MC38 cells were inoculated in the right and left flanks, respectively. The biggest tumor was treated with two doses of SFV-Nb11-Fc administered 5 days apart (3×10⁸ Viral particles/dose).

The efficacy of treatments was evaluated by measuring two perpendicular tumor diameters every 2-3 days, and tumor volumes were calculated using the formula: volume=(Length×Width²)/2. Mice were humanely sacrificed when tumor size reached ˜1200 mm³, or when tumor ulceration or evident discomfort were observed.

For rechallenge experiments in the MC38 model, mice that rejected tumors were injected s.c. with 5×10⁵ MC38 cells in the left flank three months after the first tumor inoculation. Naïve mice were included as controls and development of tumors was evaluated for two months.

Expression of Nbs from SFV Vectors In Vivo

To evaluate expression of Nbs from SFV vectors in vivo, mice were sacrificed one and five days after VP administration, and tumors, blood and urine samples were harvested to be analyzed by ELISA. Blood samples were obtained by retro-orbital venous sinus bleeding, incubated 30 min at RT and then centrifuged for 10 min at 3,000 rpm. Supernatants (i.e. serum samples) were collected for analysis. Urine samples were centrifuged at 10,000 rpm for 2 min and supernatants were collected for analysis. Tumors were weighted and homogenized in three volumes of PBS-0.05% Tween 20 supplemented with Protease Inhibitor Cocktail using a homogenizer. Samples were incubated for 1 h at 4° C. with shaking, centrifuged at 10,000 rpm for 15 min, and supernatants were collected for analysis. Total protein quantification for normalization was performed using BCA kit (Pierce, Appleton, WI), following manufacturer's instructions.

### Flow-Cytometry Analysis

Immune response after treatment with SFV vectors encoding Nb11-Fc or Nb6p-Fc was characterized in the MC38 tumor model. Draining lymph nodes and tumors were collected from each mouse five days after treatment with the different SFV vectors. Lymph nodes were homogenized in PBS using a 70 μm filter. Excised tumors were digested with 400 U/mL collagenase D and 50 μg/mL DNase-I (Roche) for 20 min at 37° C., and then homogenized with a 70 μm cell strainer. Samples were washed with PBS, centrifuged and the pellet was resuspended in PBS for staining. For functional analyses, cells were incubated with Zombie NIR Fixable viability dye (Biolegend). Then, they were blocked with anti-CD16/32 antibody (clone 2.4G2, BD Pharmingen) and stained with fluorochrome-labeled mAbs against CD45.2-APCA750 (clone 104), CD3-BV605 (clone 17A2), CD8a-BV510 (clone 53-6.7), CD4-APCAF700 (clone RM4-5), ICOS-PerCP-Cy5.5 (clone 7E.17G9), CD137-APC (clone 17B5), TIM3-BV785 (clone RMT3-23), LAG3-BV650 (clone C9B7W), CD11b-PeCy7 (Clone M1/70), CD11c-APC (clone N418), Ly6C-BV510 (clone HK1.4), F4/80-BV421 (clone BM8), PD-L1-BV785 (clone 10F.9G2) (all of them from Biolegend), PD-1-FITC (clone RMP1-30, Invitrogen), Ly6G-PE (clone 1A8, BD Biosciences) and NKp46-BV650 (clone 9-E2, BD Biosciences). For intracellular staining, cells were fixed and permeabilized with the BD Fixation/Perm buffer (BD Biosciences) and then stained with anti-Granzyme B-BV421 (clone GB11, Biolegend) and anti-KI67-PE-Cy7 (clone 16A8, BD) mAbs. To identify tumor-specific CD8⁺ T lymphocytes, cells were stained with PE-labeled H-2Kb MuLV p15E Tetramer-KSPWFTTL (MBL International, Woburn, MA). Samples were acquired on a CytoFLEX cytometer (Beckman Coulter) and data was analyzed using FlowJo software (TreeStar, Ashland, OR).

### RNA Sequencing Analysis

C57BL/6J mice bearing MC38 s.c. tumors were treated with one i.t. dose of 3×10⁸ Viral particles/tumor of SFV-Nb11-Fc or SFV-LacZ. Control untreated mice received the same volume of saline solution. Five days after treatment, mice were sacrificed, tumor samples were collected and homogenized in 2 mL of TRIzol reagent (Sigma-Aldrich). RNA was isolated following manufacturer's instructions, and further purified using the RNeasy mini kit (Qiagen). Quality of the purified RNA was evaluated using High Sensitivity RNA ScreenTape system (Agilent, Santa Clara, CA). Five samples per group with an RNA integrity number (RIN)>7 were selected and RNA samples were prepared using a TruSeq RNA Sample Prep Kit according to manufacturer's instructions (Illumina, San Diego, CA). Briefly, polyA RNAs from samples were enriched with polyT oligo-conjugated magnetic beads, fragmented and reverse transcribed using random primers. The double-stranded cDNA samples were end-repaired, adenylated and ligated to TruSeq adaptors containing the index for multiplexing. After amplifying the fragments by PCR, the library was sequenced using an Illumina NextSeq2000 (Illumina).

The workflow for RNAseq data analysis consisted in: 1) quality control of the samples using FastQC software (https://www.bioinformatics.babraham.ac.uk/projects/fastqc/), 2) alignment of reads to mouse genome (mm10) using STAR [Dobin A, et al. Bioinformatics. 2013; 29:15-21], 3) quantification of gene expression using featureCounts [Liao Y, et al. Bioinformatics. 2014], 4) gene annotation reference used was Gencode M25 [Harrow J, et al. Genome Res. 2012], and 5) differential expression statistical analysis using R/Bioconductor [Gentleman R C, et al. Genome Biol. 2004].

Gene expression data were normalized with edgeR [Robinson M D, et al. Bioinformatics. 2010]. Genes with read counts lower than six in more than 50% of the samples were not considered in this experiment. LIMMA [Ritchie M E, et al. Nucleic Acids Res. 2015] was used to identify the genes with significant differential expression between the experimental conditions, using a cut-off of p<0.05. Further functional analysis using Gene Set Enrichment Analysis (GSEA) with the MsigDB C7 collection of gene sets were performed [Subramanian A, et al. Proc Natl Acad Sci. 2005]. For this analysis, p<0.01 and false discovery rate (FDR) <0.05 were considered statistically significant.

### Non-Viral Delivery of SFV by Electroporation

Six-week-old female C57BL/6J mice were challenged with 5×10⁵ MC38 cells s.c. in the right flank. After ten days, intratumoral delivery of pBK-T-SFV plasmids harboring SFV replicons containing Nb11-Fc or LacZ genes was performed by electroporation as described previously (Silva-Pilipich N, et al, Molecular Therapy Nucleic Acids, 2022), with some minor changes. Briefly, two hours before treatment hyaluronidase type IV (Sigma) was injected i.t. (30 units/tumor). 20 μg of pBK-T-SFV endotoxin-free plasmid/dose was used, diluted in 25 μL of PBS. Mice were anesthetized, plasmid was injected i.t. and, immediately after that, local electroporation was performed using the ECM 830 electroporation system (BTX, Holliston, MA) and the following conditions: eight pulses of 1200 V/cm of 0.1 ms duration each, separated by 5 ms. For the first round of treatment, tumors were exposed performing a simple surgery. This procedure was performed three times every three days.

### Statistical Analysis

Data are expressed as the mean±SD or mean±SEM, as specified in each figure legend. Prism software (GraphPad Software, San Diego, CA) was used for statistical analysis. To compare multiple experimental groups, one-way ANOVA test and Tukey's multiple comparison test were used. Two-tailed Student's t test was applied to compare two experimental groups. For time-series analysis, data were compared using the extra sum-of-squares F test and fitted to a second-order polynomial equation. Survival of tumor-bearing mice is represented by Kaplan-Meier plots and analyzed by log-rank test. The p values <0.05 were considered statistically significant.

### Results

### Characterization of PD-1 and PD-L1-Specific Nb

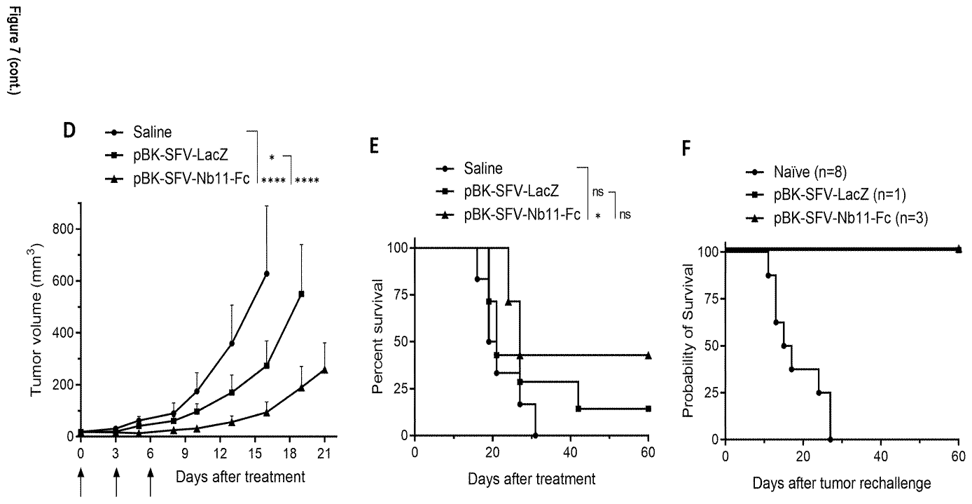

To evaluate the ability to inhibit binding of PD-L1 to PD-1, we performed an inhibition ELISA using commercially available PD-1 and PD-L1 ectodomains fused to human IgG1 Fc domain. Nb11 and Nb6p nanobodies were able to inhibit binding of human PD-1 to PD-L1 (FIGS. 8A, B). We also evaluated the ability of these Nbs to inhibit the binding of murine orthologs in a similar inhibition ELISA and found that these two nanobodies able to inhibit PD-1/PD-L1 binding by more than 50% at 300 nM (FIG. 8C). Nb6p showed in fact a markedly superior inhibition capacity, comparable to an anti-mouse PD-L1 antibody that had shown potent antitumor activity in previous work (FIG. 8D) [Ballesteros-Briones M C, et al, Mol Ther. 2019].

### Evaluation of SFV Vectors Encoding Monomeric Nbs Against PD-1 and PD-L1

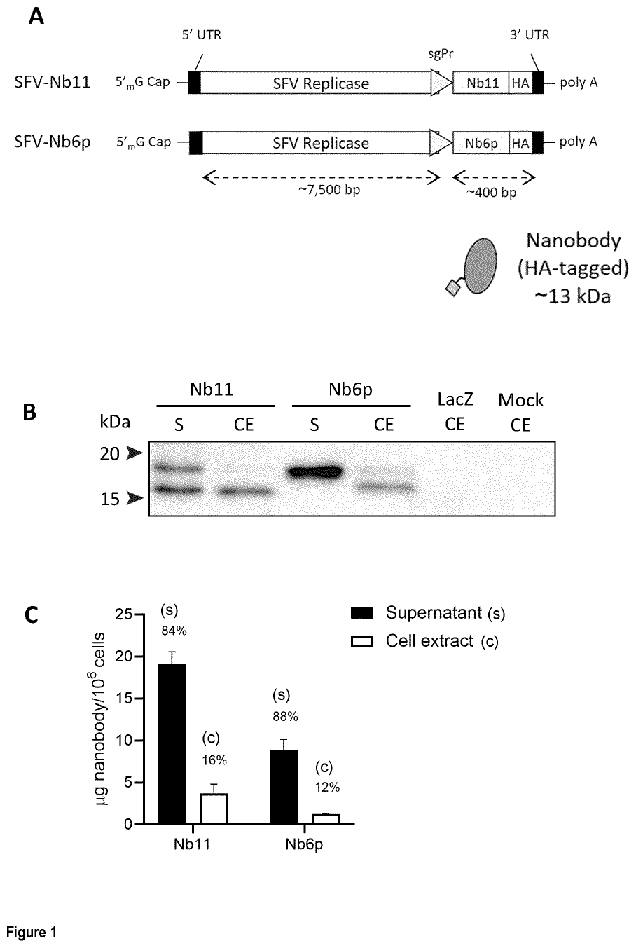

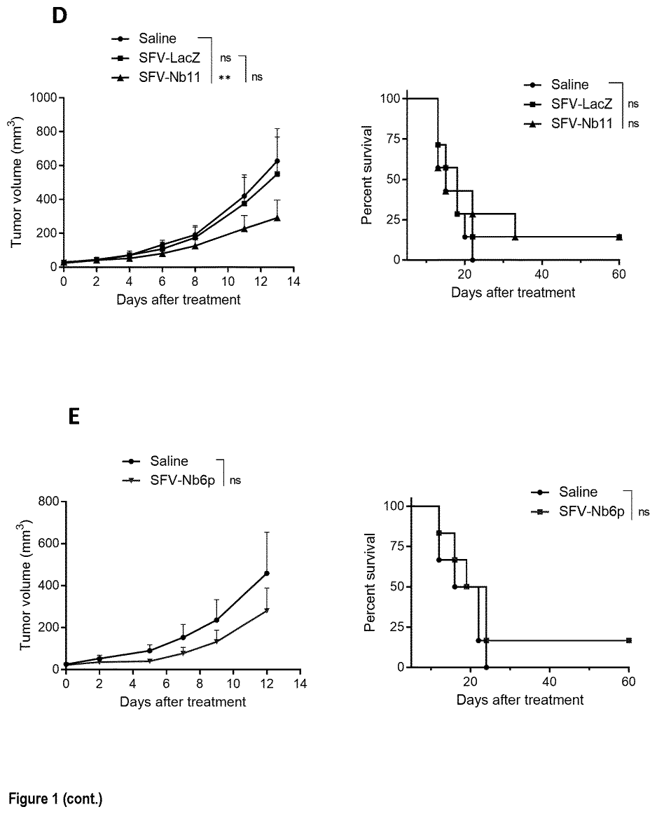

In this work we have developed SFV vectors expressing both Nb11 and Nb6p Nbs in order to test their antitumoral potential by local delivery into tumors. For that purpose, we cloned the Nb11 and Nb6p sequences into SFV, generating SFV-Nb11 and SFV-Nb6p vectors, respectively. In both cases the Nb sequence was designed with a signal peptide at the amino terminus and a hemagglutinin tag at the carboxyl terminus for detection (FIG. 1A). Nb expression was evaluated in BHK-21 cells infected with SFV-Nb11 and SFV-Nb6p viral particles at 24 h post-infection. SFV-LacZ vector was used as control. Immunofluorescence (IF) analysis of infected cells showed co-expression of both Nb and SFV replicase (results not shown). Western blot analysis showed that both Nbs were expressed with the expected size (around 15 kDa) in cell extracts but showed a higher molecular weight (MW) in the supernatants (FIG. 1B). This is in line with previous observations for Nb11 expressed from an AAV vector plasmid, in which we demonstrated that this shift was due to glycosylation and that it did not affect antigen-binding [18]. Quantification of Nbs was done by a specific ELISA against PD-1 or PD-L1, and in both cases we confirmed that the secretion was very effective, as the majority of the Nb (˜85%) was present in supernatants. Expression levels were around 20 and 10 μg of secreted Nb11 and Nb6p, respectively, per 10⁶ infected cells (FIG. 1C).

We then evaluated the antitumor activity of SFV-Nb11 and SFV-Nb6p vectors in a colon adenocarcinoma mouse model using MC38 cells implanted subcutaneously. Tumors were treated with 3×10⁸ Viral particles intratumorally, a dose that had previously shown a significant antitumor effect in this model using a SFV vector encoding a full-length anti-PD-L1 mAb (SFV-aPDL1) [Ballesteros-Briones M C, et al, Mol Ther. 2019]. An antitumor effect was achieved using SFV-Nb11 and SFV-Nb6p vectors in vivo and some delay in the rate of tumor growth was observed (FIG. 1D, E).

### Generation of SFV Vectors Expressing Optimized Nbs

SFV vectors expressing Nb11 and Nb6p were modified by fusing each Nb sequence to an IgG Fc domain to promote their dimerization. For this purpose, Nb11 was fused to mouse IgG1 Fc domain (generating SFV-Nb11-Fc), an isotype with no secondary functions as PD-1 is expressed mainly on T cells (FIG. 2A). Nb6p was fused to mouse IgG2a Fc domain, generating SFV-Nb6p-Fc (FIG. 2A).

Expression of the Nb-Fc fusion molecules was first analyzed in BHK-21 cells infected with SFV-Nb11-Fc and SFV-Nb6p-Fc vectors. In this experiment we used as controls SFV vectors expressing conventional mAbs against murine PD-L1 and PD-1 (SFV-aPD1 and SFV-aPDL1 [Ballesteros-Briones M C, et al, Mol Ther. 2019], respectively). As observed before, infected cells showed co-expression of both Nb-Fc and SFV replicase by IF (results not shown). Quantification of recombinant antibodies from infected cells was performed by ELISA, observing a significantly higher expression for Nb-Fc molecules compared to mAbs (FIG. 2B). The expression levels of dimeric Nbs were two to four-fold lower than those observed for the monomeric forms (FIG. 1C), although they were also efficiently secreted (FIG. 2B). Both Nb11-Fc and Nb6p-Fc were able to dimerize when analyzed by Western blot using non-reducing conditions (FIG. 2C, lower panel). Under reducing conditions, both Nb-Fc molecules showed the expected MW (approximately 40 kDa, FIG. 2C, upper panel).

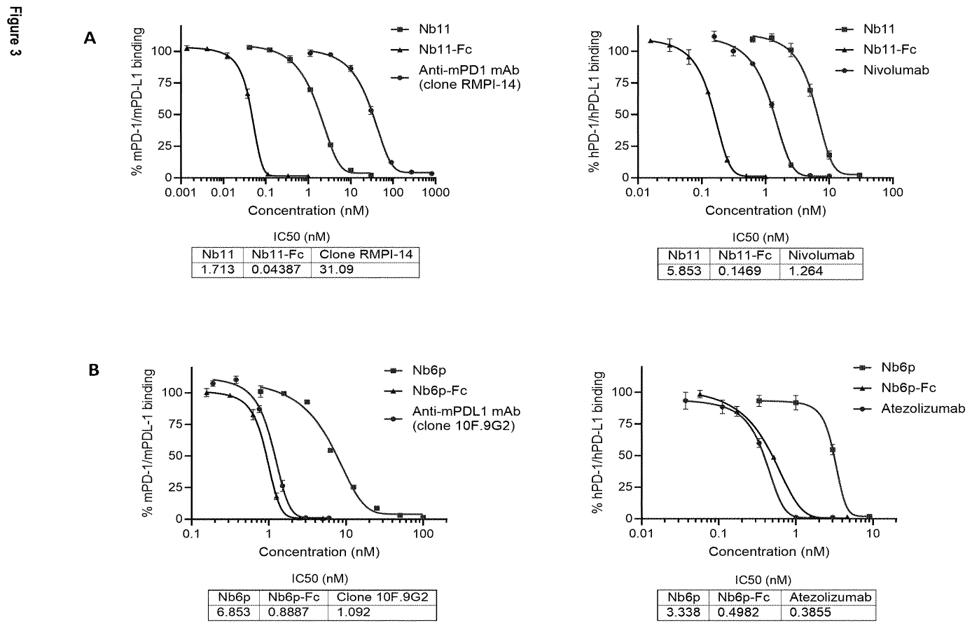

The fusion of Fc domains to Nb could have an impact on their antigen-binding properties, as dimerization could increase their avidity. To test whether this was the case, inhibition curves for PD-1/PD-L1 binding were generated using monomeric Nbs and the Fc-fused versions, which were previously purified from SFV-transfected cells as described in methods. Interestingly, fusion to Fc domains improved the inhibition potential of Nbs for mouse and human molecules with an approximate 40- and 7-fold IC50 reduction for Nb11-Fc and Nb6p-Fc, respectively (FIG. 3). We also included commercially available mAbs in these assays and observed that Nb6p-Fc had similar IC50 compared to both atezolizumab and clone 10F-9G2, an anti-mouse PD-L1 mAb commonly used in preclinical studies [Grasselly C, et al, Front Immunol. 2018]. In the case of Nb11-Fc, we observed a 10- and 700-fold IC50 reduction when compared with nivolumab and clone RMPI-14, respectively. This last clone is an anti-mouse PD-1 mAb which has shown potent antitumor activity in preclinical studies [Grasselly C, et al, Front Immunol. 2018; Ngiow S F, et al Cancer Res 2015].

### Antitumor Activity of SFV Vectors Expressing Nb-Fc

Since Nb-Fc fusions led to a decrease in the IC50 values for PD-1/PD-L1 inhibition, we reasoned that these molecules could have a better performance than monomeric Nbs in vivo. SFV vectors encoding Nb11-Fc and Nb6p-Fc were evaluated in the MC38 subcutaneous tumor model, including vectors encoding monomeric Nbs (SFV-Nb11 and SFV-Nb6p) and conventional mAbs (SFV-aPD1 and SFV-aPDL1) for comparison. SFV-Nb11-Fc and SFV-Nb6p-Fc showed a very potent antitumor effect, delaying tumor growth and significantly improving survival compared to control groups (saline and SFV-LacZ) (FIG. 4, left panels). Treatment with SFV-Nb11-Fc and SFV-Nb6p-Fc led to 53% and 60% long-term survival, respectively, in contrast to 20% and 28% obtained with SFV-aPD1 and SFV-aPDL1, respectively (FIG. 4, middle panels). As observed before, monomeric Nbs showed a modest antitumor effect, which was similar or lower to that obtained with conventional antibodies. Treated animals that had complete remissions remained tumor-free after being rechallenged with MC38 cells (FIG. 4, right panels), suggesting that these treatments were able to generate an efficient memory immune response.

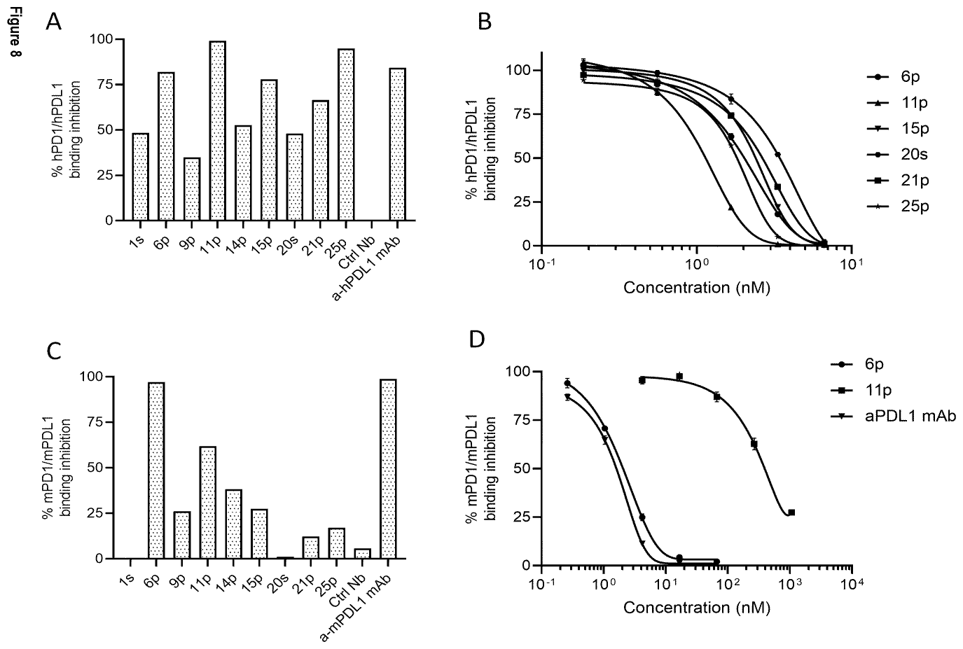

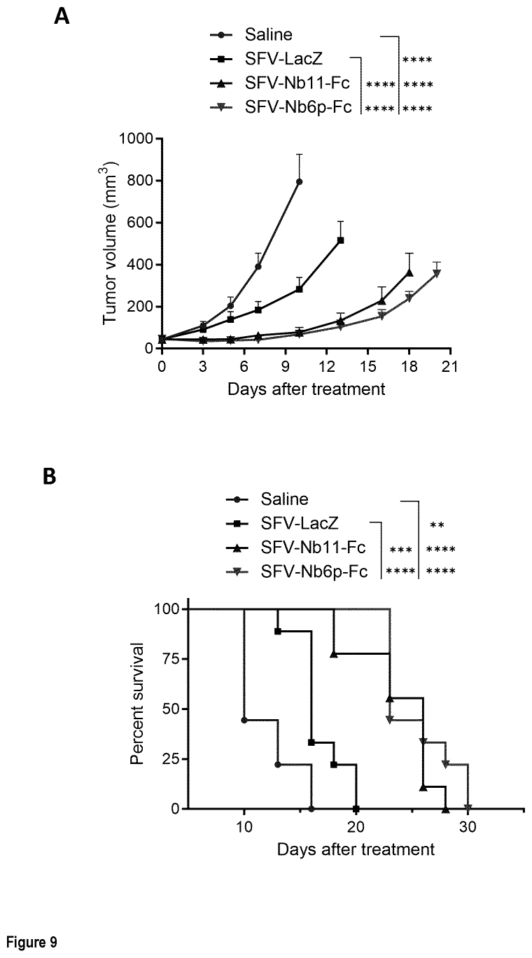

The efficacy of SFV-Nb11-Fc and SFV-Nb6p-Fc was also validated in a melanoma tumor model (B16-OVA), where a significant delay in tumor growth and improvement in survival was observed in comparison with saline and SFV-LacZ groups (FIG. 9).

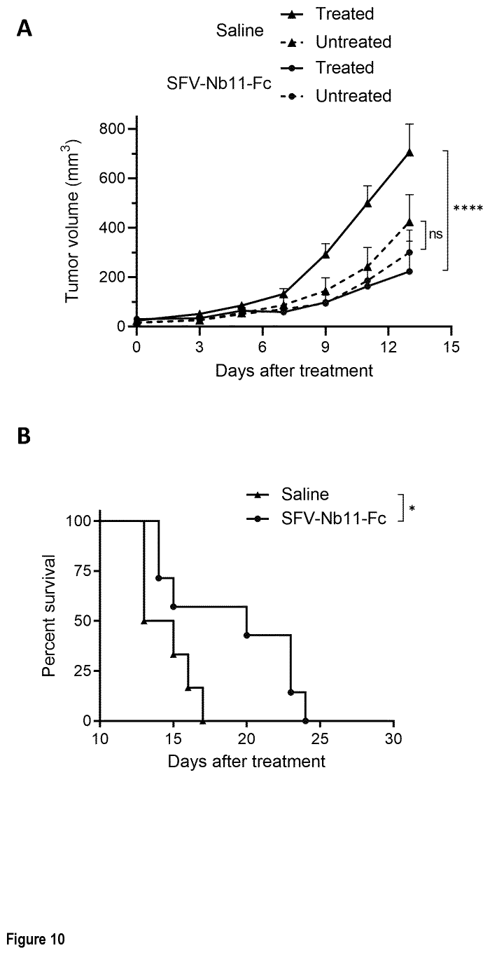

One of the main challenges for local immunotherapies is to trigger systemic antitumor effects. To evaluate whether SFV expressing dimeric Nbs could lead to abscopal effects in non-treated tumors, we used a bilateral subcutaneous MC38 tumor model. Mice received two intratumoral doses of SFV-Nb11-Fc vector (3×10⁸ Viral particles/dose), five days apart, in one of the nodules. Although the vector led to a significant reduction in the size of the treated tumor, it had a more modest effect in terms of controlling the growth of non-treated nodules. Untreated tumors from mice that received SFV-Nb11-Fc showed a slight delay in growth from day nine after the first dose onwards compared to control mice that only received saline, however, differences were not significant (FIG. 10A). Nevertheless, treating only one tumor nodule with SFV-Nb11-Fc was enough to significantly increase the survival of mice compared to controls (FIG. 10B), suggesting that this kind of therapy could induce abscopal effects.

In Vivo Expression of Nbs from SFV Vectors

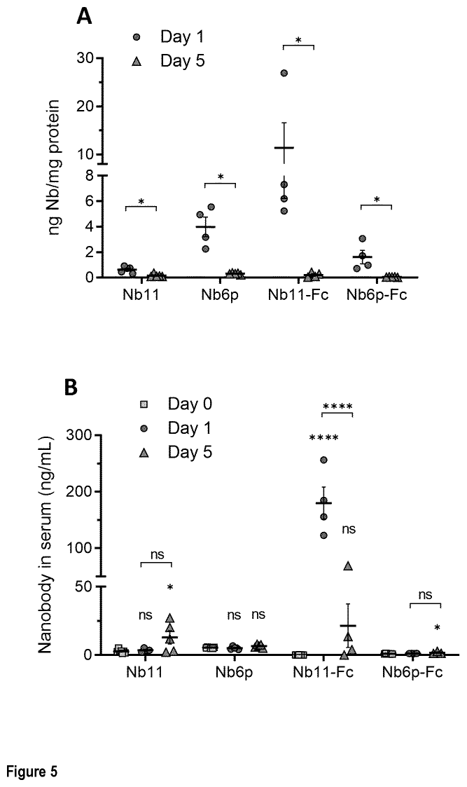

We then evaluated the levels and persistence of the different Nbs expressed from SFV administered locally in MC38 tumors using the same dose as in therapeutic experiments. We only observed expression of monomeric and Fc-fused Nbs in tumor tissue on day one post-treatment, while on day five Nb levels were undetectable in all mice (FIG. 5A). This is in line with previous observations using SFV vectors. In most cases Nbs were not detected in blood, but a significant leakage to systemic circulation was observed for Nb11-Fc on day one post-injection, with levels around 180 ng/ml (FIG. 5B). This might be due to the higher intratumoral expression obtained for this Nb compared to Nb6p-Fc, or to the fact that anti-PD-L1 Nbs could be retained more efficiently in the tumor microenvironment than anti-PD-1 Nbs due to the high PD-L1 expression in MC38 tumors [10]. In the case of monomeric Nbs, we were also able to detect them in urine, with a slight increase at day one, suggesting that these small molecules could be eliminated from circulation through renal clearance (FIG. 5C).

### Antitumor Immune Responses Elicited by SFV Encoding Dimeric Nbs

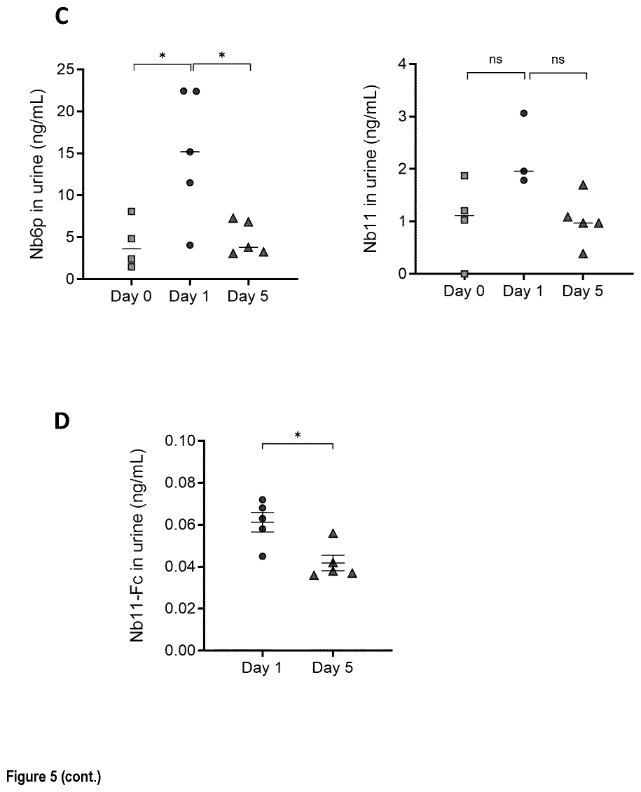

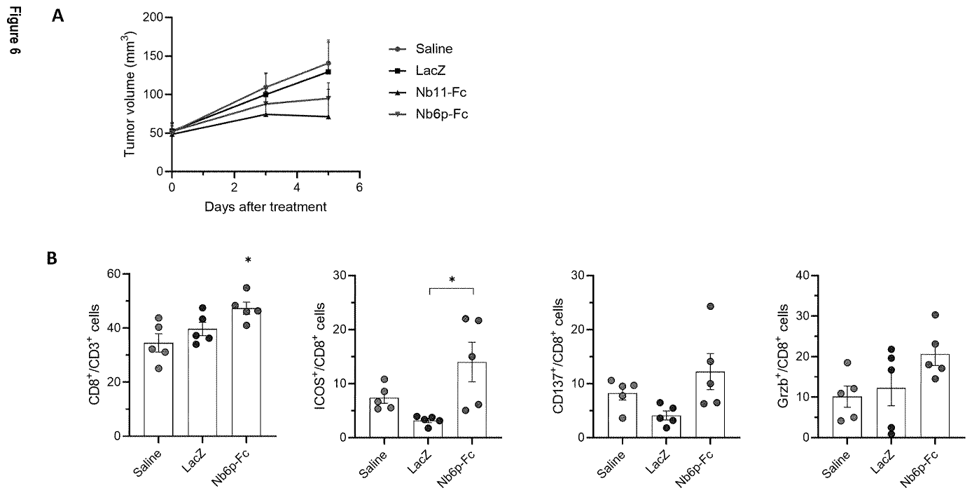

To evaluate the antitumor immune responses elicited by SFV vectors encoding Nb-Fc nanobody conjugates, mice bearing MC38 tumors were treated with SFV-Nb11-Fc and SFV-Nb6p-Fc vectors, as well as with SFV-LacZ and saline as controls, and sacrificed five days later. Tumor and draining lymph nodes (dLNs) samples were processed and analyzed by flow-cytometry using different markers. In the case of SFV-Nb11-Fc, mice had very small tumors at the moment of sacrifice and could not be included in this analysis (FIG. 6A). However, we did analyze changes in dLNs for both Nb groups.

A significant increase of total CD8+ T cells was observed in tumors treated with SFV-Nb6p-Fc, which expressed higher levels of activation marker ICOS and a trend to express higher levels of CD137 and granzyme b (FIG. 6B). Although no relevant changes were observed in total MuLV tetramer-specific CD8⁺ T cells in the SFV-Nb6p-Fc group, these cells showed a significant increase of the exhaustion marker TIM-3, suggesting a stronger activation state in this group (FIG. 6C). No significant changes in PD-1 expression were observed in total and MuLV-specific CD8+ T cells in tumors (FIG. 6D), as well as no global changes in the CD4⁺ T cell population (data not shown). A higher percentage of NK cells (NKp46+ cells) and CD11b⁺ cells were found in tumors treated with SFV-Nb6p-Fc compared to saline and SFV-LacZ groups (FIG. 6E). However, no changes were seen for macrophages (F4/80+CD11b⁺ cells), while there was a significant decrease in granulocytes (Ly6G+CD11b⁺ cells) (FIG. 6E).

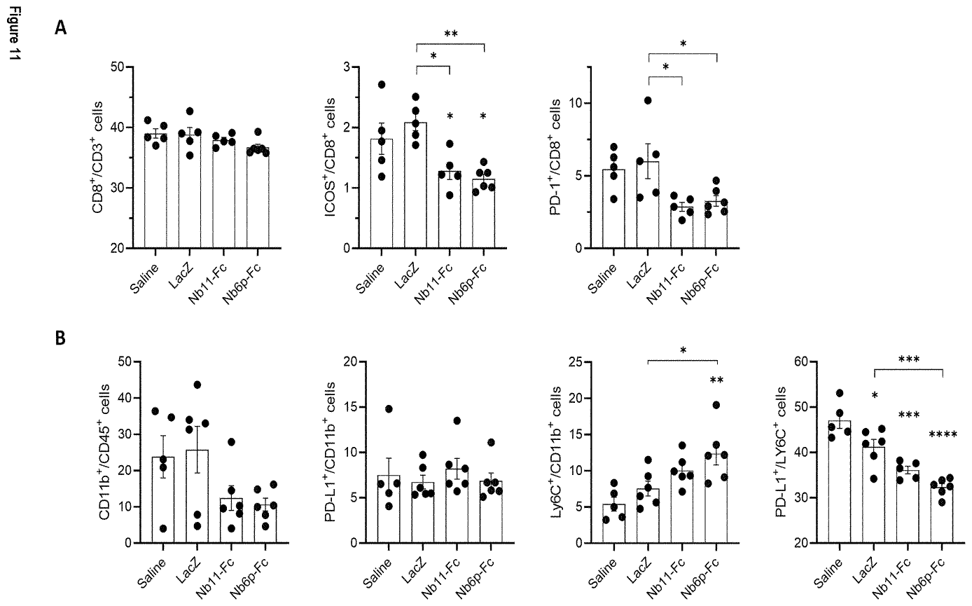

In dLNs, although no global changes were observed for total CD8⁺ T cells, this population showed significant lower levels of PD-1 in mice treated with both SFV-Nb-Fc vectors, which might indicate a less exhausted phenotype in this location (FIG. 11A). These cells, however, showed lower level of ICOS. In contrast to what was observed in tumors, CD11b⁺ cells in dLNs showed a tendency to be decreased in response to both Nb-Fc treatments with no changes in PD-L1 (FIG. 11B). A significant increase in Ly6C⁺CD11b⁺ cells was observed for SFV-Nb6p-Fc, and SFV-Nb11-Fc also showed a similar trend (FIG. 11B). Interestingly, these cells expressed lower levels of PD-L1 in both Nb-Fc groups.

Tumor samples from animals treated with SFV-Nb11-Fc, SFV-LacZ, and saline were used for bulk RNA sequencing analysis to gain a deeper insight into immunological altered pathways. Several immune-related genes were found to be differentially expressed in tumors treated with SFV-Nb11-Fc compared to tumors injected with saline. SFV-LacZ vector was also able to induce important changes in the transcriptome in three out of five analyzed tumors, underlying the significance of SFV vector in immune modulation. However, changes were more homogenous and therefore more significant for tumors treated with SFV-Nb11-Fc. As expected, several viral-stress inducible genes (Sting1, Batf2, Nod1, Tirap, Ikbke, Clec4d, Clec4e, Irf1) were found upregulated in SFV-treated tumors. In addition, SFV-Nb11-Fc treatment generated an upregulation of different immunostimulatory genes, including cytokines (Il2g, Il2ra, Il15ra, Il18rap, Ifng, Tgfa), chemokines (Cxcl9, Cxcl10, Cxcl11), and IFN-I response genes (Stat1). The upregulation of molecules involved in cell adhesion and motility (such as Icam1, Itgal, Selp), suggests an increase in the recruitment of immune cell populations to the tumor site. Several genes associated with enhanced activity of NK cells and cytotoxic CD8⁺ T cells (Ncr1, Nkg7, Prf1, Gzma, Gzmb, Gzmk), and genes expressed upon TCR stimulation (Nfatc2, Lat, Cd6, Cd5) were also upregulated, suggesting an increase in the infiltration and activation of these effector cells. Increased expression of genes related with apoptosis (Cd40, Cd40lg, Fasl, Ripk1) was also observed. Finally, SFV-Nb11-Fc treatment led to a downregulation of genes related with angiogenesis (Vegfa and Jmjd8), and pro-metastatic factors (Cxcr4, Mmp11, Macc1). The later are known markers of poor prognosis in different cancer types, including colorectal cancer.

Transcriptome differences between tumors treated with SFV-Nb11-Fc and SFV-LacZ were analyzed more extensively using the gene set enrichment analysis (GSEA) tool [Subramanian A, et al, Proc Natl Acad Sci 2005] and the C7 database, to compare changes in the immunologic signatures. Important differences were found for both treatment groups, and many of the altered gene sets were related to CD8⁺ T cell activation, NK function, and IFNγ signalization in different cells.

### Non-Viral Delivery of SFV Vector Encoding Anti-PD-1 Dimeric Nb

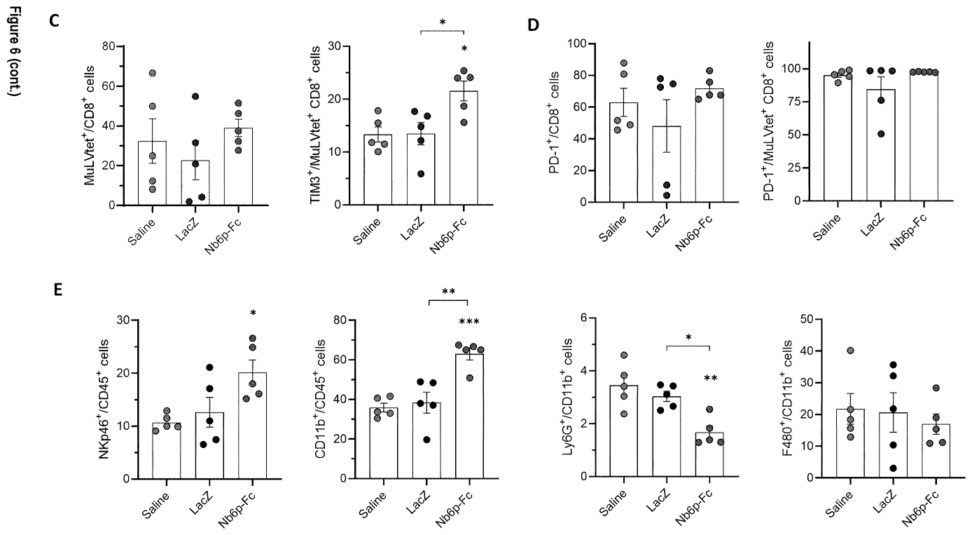

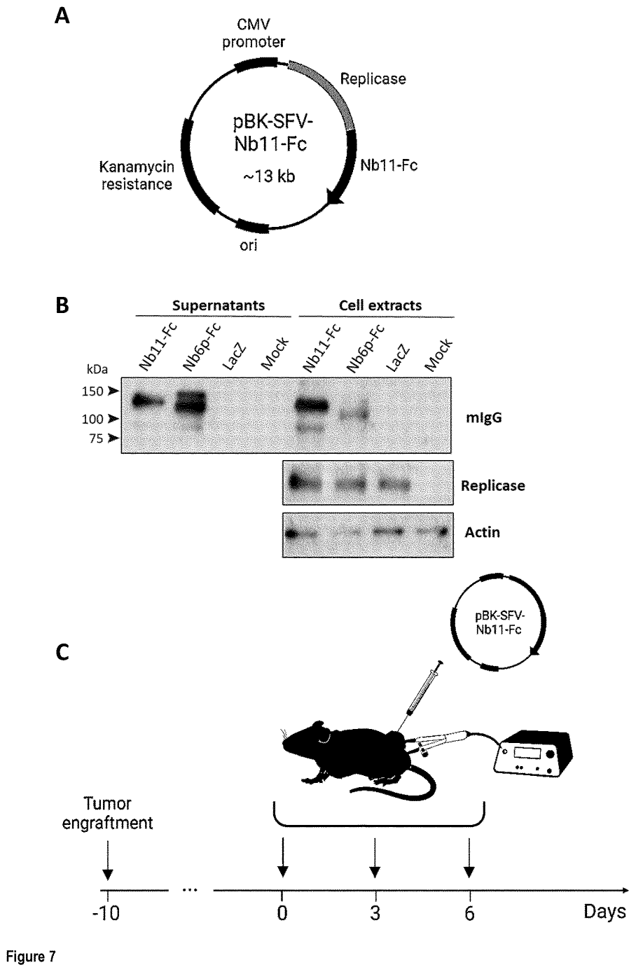

An alternative to use of SFV Viral particles would be to use a non-viral approach to deliver SFV vectors into tumors. To test whether this delivery system could be used for SFV vectors expressing dimeric Nbs, we generated a plasmid having the SFV-Nb11-Fc replicon under the transcriptional control of the CMV promoter (pBK-SFV-Nb11-Fc) (FIG. 7A). Expression of Nb11-Fc from this plasmid was confirmed in vitro by Western blot analysis of transfected BHK-21 cells (FIG. 7B). As schematized in FIG. 7C, mice bearing MC38 subcutaneous tumors received three doses of pBK-SFV-Nb11-Fc plasmid (20 μg/dose) every three days, followed by local electroporation as described previously (Silva-Pilipich N, et al, Molecular Therapy Nucleic Acids, 2022). This treatment protocol led to a significant reduction in tumor growth compared to untreated controls or mice that received SFV-LacZ plasmid by electroporation (FIG. 7D). A significant increase in survival for animals that received pBK-SFV-Nb11-Fc compared to untreated mice was also achieved (FIG. 7E). Similar to mice treated with viral vectors, this strategy led to 100% of protection after MC38 tumor rechallenge, suggesting that this non-viral delivery system is also able to promote long-lasting antitumor immune responses.

### Discussion

Antibodies able to block the PD-1/PD-L1 axis, such as nivolumab, pembrolizumab, and atezolizumab, are showing remarkable therapeutic effects in patients with different types of tumors [Xiang Z, et al, Front Pharmacol. 2022]. However, frequent adverse effects observed in patients treated with checkpoint inhibitors, as well as the lack of responses in some tumor types, makes necessary the improvement of these therapies [Sun G, et al, Int J Oncol. 2022]. In this work, we have addressed these issues by developing a strategy based on local intratumoral delivery of Nbs using a self-amplifying RNA vector that can induce type I IFN responses and apoptosis in tumor cells, favoring responses in tumors that usually do not respond well to immunotherapy. We have developed new Nbs against PD-L1 (Nb6p) and PD-1 (Nb11) able to block the interactions of PD-1 and PD-L1 in both mouse and human molecules. The blocking efficacy of Nb6p and Nb11 was improved by homo-dimerization using an IgG Fc domain, which resulted in a considerable IC50 reduction in PD-1/PD-L1 binding assays (approximately 8- and 40-fold reduction for Nb6p and Nb11, respectively, for both murine and human interactions). Remarkably, Nb11-Fc showed an IC50 that was 8.6-fold lower than nivolumab, and Nb6p-Fc had an IC50 very similar to atezolizumab. These results indicate that these newly described dimeric Nbs could have clinical potential as recombinant proteins. A similar anti-PD-L1 camel-derived Nb fused to a human IgG1 Fc, named envafolimab, has been recently approved for the treatment of various solid tumors in China, and is tested for soft tissue sarcomas and biliary tract cancer in the USA [Markham A. Drugs 2022]. Interestingly, envafolimab is administered subcutaneously and has demonstrated a high safety profile. Envafolimab has shown an IC50 of 5.25 nM for blocking the PD-L1 the PD-1 interaction [Markham A., Drugs 2022], which is about 10-fold higher than the one we have observed with Nb6p-Fc.

Although dimeric Nbs are larger than their corresponding monomeric forms, their size is approximately half of conventional antibodies, which could endow them with a better penetrability into tumors. An additional advantage of dimeric Nbs is the possibility to modulate their functions using different Fc domains.

The improved blocking activity shown by dimeric Nbs in vitro was recapitulated in vivo when expressed from SFV vectors delivered intratumorally. In fact, SFV vectors expressing monomeric Nbs already showed antitumor activity in MC38 colon adenocarcinoma tumors. SFV vectors expressing either Nb11-Fc or Nb6p-Fc had a potent antitumor effect in both colon and melanoma murine tumors, leading to more than 50% complete regressions in the first model with both vectors. This effect was better than the one obtained with SFV vectors expressing anti-PD-L1 and anti-PD-1 conventional mAbs, although these vectors also showed significant antitumor effects, as had been previously described [Ballesteros-Briones M C, et al, Mol Ther. 2019]. One possible reason for the better performance of SFV vectors harboring dimeric Nbs could be the much higher expression of these molecules compared to mAbs, as it was observed in vitro in infected cells (FIG. 2C). Nb expression in vivo was very transient in all vectors and was detected mainly one day after treatment. Although expression seemed to be restricted to tumors, in the case of Nb11-Fc a relatively high level was transiently observed in serum, suggesting that part of the dimeric Nb was able to leak out of the tumor. However, this effect was not observed for Nb6p-Fc, maybe due to the high expression of PD-L1 in MC38 tumors. A low level of monomeric Nbs was observed in urine one day after vector administration. Although in our study Nbs were expressed locally, it was also expected that the fraction leaking out of the tumor would be eliminated rapidly through urine, as it seems to be the case, since very low plasma levels of monomeric Nbs were detected. However, dimeric Nbs were detected at very low levels in urine, confirming that the Fc domain could increase their half-life in serum.

A remarkable feature of this SFV-based therapy is that a single dose of the vector expressing locally the dimerized Nb for a very short period of time was able to promote potent and long-lasting antitumor responses. A number of immunostimulatory genes were significantly upregulated in tumors treated with SFV-Nb11-Fc compared to saline control group. The expression of dimerized Nbs able to block the PD-1/PD-L1 axis seemed to be crucial to trigger a curative immune response.

SFV viral particles can be produced quite efficiently and a recent clinical trial in cervical cancer patients with SFV vectors expressing human papilloma (HPV) virus E6 and E7 proteins has demonstrated that they can have a high degree of safety in humans [Komdeur F L, et al, Mol Ther. 2021]. However, clinical implementation of viral particles can be more difficult compared to the use of non-viral vectors, which are less expensive to produce at GMP levels and could have a higher safety profile. The SFV system has the advantage that it can also be used in a non-viral mode, either as RNA or as a DNA/RNA layered plasmid vector. We have chosen this last option to evaluate one of the two SFV vectors expressing dimerized Nbs (pBK-SFV-Nb11-Fc). Interestingly, local delivery of this plasmid into MC38 tumors by electroporation resulted in antitumor effects comparable to those obtained with viral particles. An additional advantage of the DNA system is that no neutralizing antibodies against SFV will be induced, allowing for multiple administrations.

### Example 2. Immunoconjugates Containing Anti-PD-1 and Anti-PD-L1 Nanobodies Fused to Interleukine 12

### Construction of SFV Vectors Expressing Fusion Proteins of IL-12 and Nanobodies Nb6p and Nb11

In order to achieve a greater antitumor effect, we constructed SFV vectors that express IL-12 fused to Nb11 and Nb6p nanobodies (Nbs) according to the schemes shown on FIG. 12. To do this, a single-chain version of IL-12 (scIL 12) [G. J. Lieschke, et al, Nat. Biotechnol. 15 (1997)] has been used with each nanobody fused at the amino or carboxy-terminal end, generating four SFV vectors expressing the following fusion proteins: scIL 12-Nb11, scIL 12-Nb6p, Nb11-scIL 12, and Nb6p-scll12 (FIG. 12A). A different type of constructs was based on a double-chain version of IL-12 (dcIL12) [Rodriguez-Madoz J R, et al, Mol Ther. 2005] in which each subunit of IL-12 (p35 or p40) is fused with a Nb (in this case, generating an IL-12 with two copies of the Nb). In this case, three SFV vectors expressing the following fusion proteins have been generated: dcIL 12-Nb11, dcIL 12-Nb6p, and dcIL 12-bis (this one contains p35 fused to Nb6p and p40 fused to Nb11) (FIG. 12B).

### Activity of ICKs In Vitro

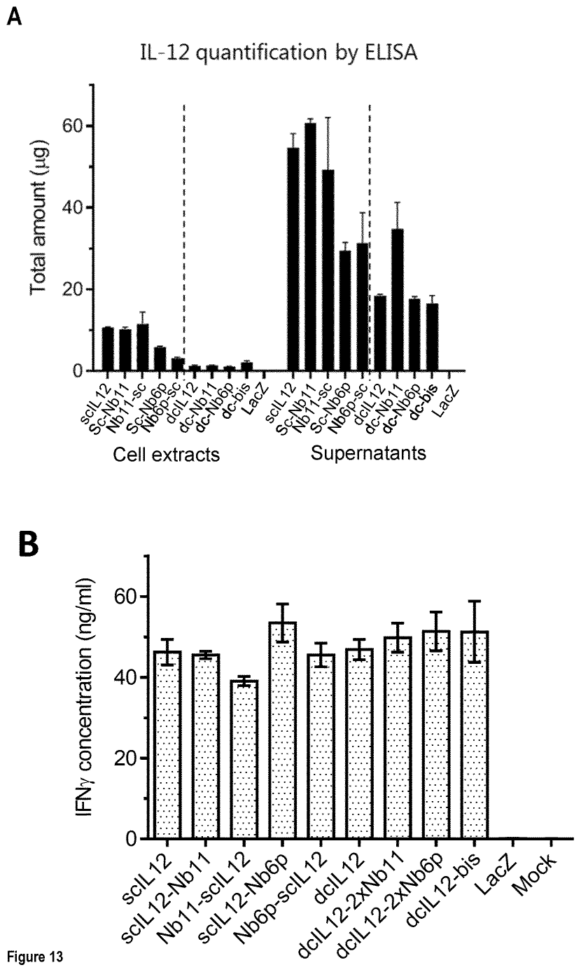

In order to test the activity of ICKs we generated Viral particles for each of the SFV vectors described previously. We then infected BHK cells with Viral particles from each SFV vector at a multiplicity of infection (MOI) of 10, using cells infected with the same amount of SFV-LacZ [Quetglas J I, et al, Gene Ther. 2012] as a negative control. In this assay we also used SFV vectors expressing scIL 12 and dclL 12 as positive controls [Rodriguez-Madoz J R, et al, Mol Ther. 2005]. We collected supernatants and cell extracts 24 h later and the amount of IL-12 was quantified using a commercial ELISA kit (BD Biosciences). As shown in FIG. 13A, SFV-scIL 12-Nb11 and SFV-Nb11-scIL 12 expressed similar levels of IL-12 as SFV-scIL 12 (approximately 50 μg/10⁶ cells), while SFV-scIL 12-Nb6p and SFV-Nb6p-scIL 12 showed lower expression levels (approximately 30 μg/10⁶ cells). In the case of vectors expressing dcIL 12-Nb fusions, all of them expressed levels similar to those of control SFV-dcIL 12 vector (approximately 20 μg/10⁶ cells), except SFV-dcIL 12-Nb11, which showed higher levels of expression (approximately 40 μg/10⁶ cells).

In order to test the activity of IL-12 fusion proteins we extracted and isolated splenocytes from healthy mice. Animals were sacrificed and the spleen collected in 10 ml PBS or unsupplemented RPMI 1640 medium. Tissue disintegration was performed with a cell strainer. The final pellet was resuspended in 1 ml RPMI medium supplemented with 10% FBS and antibiotics. 2×10⁶ splenocytes were plated/well of M24 plates diluted in 1 ml RPMI medium. Supernatants from cells infected with SFV-ICK Viral particles were incubated with mouse splenocytes in order to stimulate IFNγ production. Incubation was carried out for 48 h at 37° C. Supernatant from cells infected with SFV-IL-12 PV was used as a positive control and supernatants from untransfected BHK cells or complete RPMI 1640 medium as a negative control. After incubation, the samples were centrifuged for 5 min at 6000 rpm, supernatants were collected and quantified by commercial IFNγ-specific ELISA (Mouse IFNγ ELISA Set, BD Biosciences). As observed in FIG. 13B, all ICKs induced similar levels of IFNγ, indicating that all of them are equally active.

### Activity of ICKs In Vivo

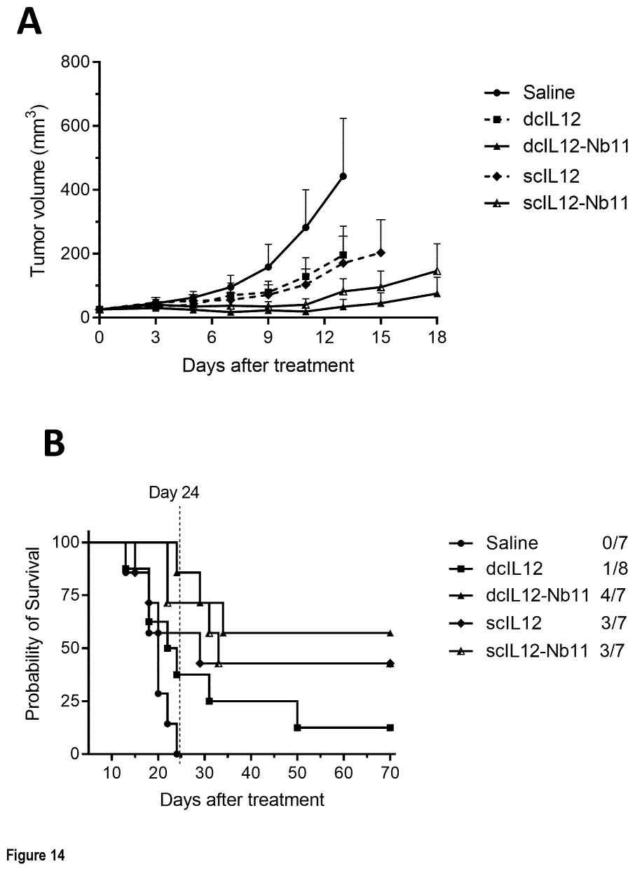

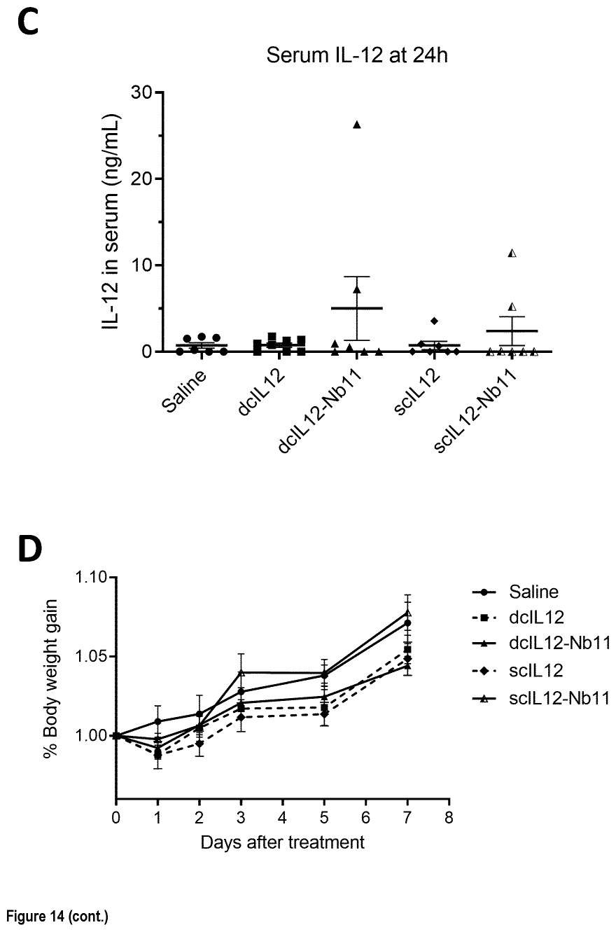

C57BL/6J mice were subcutaneously injected in the right flank with 5×10⁵ tumor cells diluted in saline solution. Seven-ten days after tumor inoculation, SFV vectors were administered intratumorally in a total volume of 50 μL, diluted in saline solution. In a first experiment, and in order to compare the antitumor activity of the different vectors, a dose of 2×10⁷ Viral particles/tumor was used. In this experiment we included the following vectors: SFV-scIL 12-Nb11, SFV-dcIL 12-Nb11, and SFV-dcIL12-bis. SFV-scIL 12 and SFV-dcIL 12 were also included to be compared with the ones expressing ICKs. Control untreated mice received the same volume of saline solution. As can be observed in FIGS. 14A and 14B SFV vectors having Nb11 fused to either scIL 12 or dcIL 12 showed a high antitumor response, leading to 42% and 57% long-term survival, respectively. In the case of SFV-dcIL 12-Nb11, the response was much higher than that of SFV-dcIL 12, which only led to 12.5% long-term survival. For SFV-scIL 12-Nb11 the response was similar to SFV-scIL 12 although the first vector was able to control better the tumor growth (FIG. 14A). We also measured IL-12 levels in serum 24 h after treatment. Most mice did not show IL-12 in blood, with very low levels detected in four mice, each of them belonging to a different group (FIG. 14C). No toxicity was observed in any of the groups, since no loss of body weight could be detected (FIG. 14D).

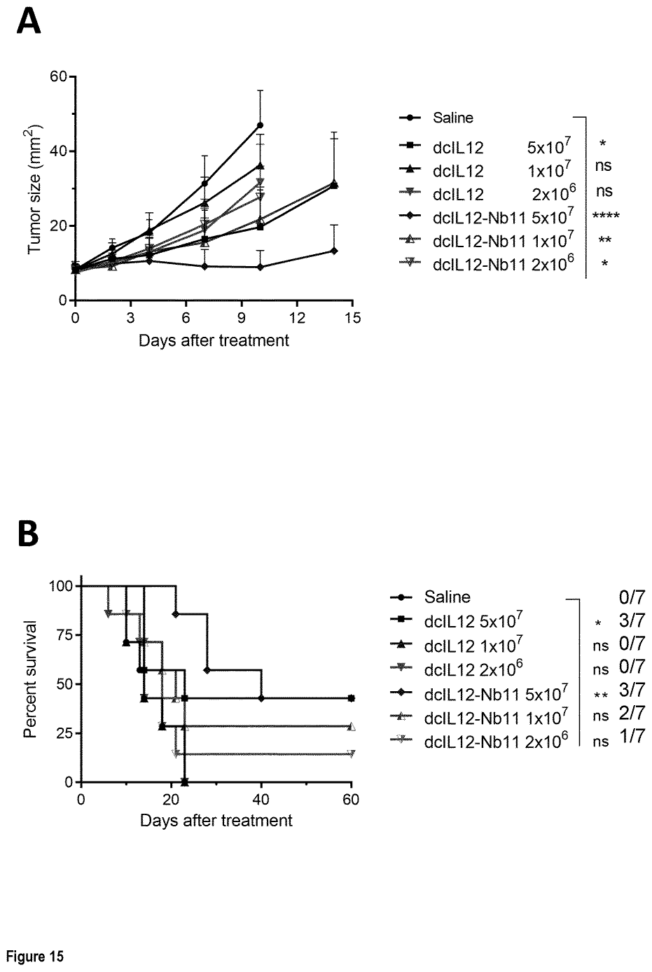

Since SFV-dcIL 12-Nb11 showed surprising antitumor effects, we performed a dose range finding study comparing it with SFV-dcIL 12. For this purpose, we treated MC38 tumors as described before with three doses of each vector: 2×10⁶, 1×10⁷, and 5×10⁷ Viral particles. As shown in FIGS. 15A and 15B, SFV-dcIL 12-Nb11 was more efficient than SFV-dcIL 12 at all tested doses. For example, 1×10⁷ VP of SFV-dcIL 12-Nb11 induced a similar tumor growth inhibition effect than 5×10⁷ Viral particles of SFV-dcIL 12 (FIG. 15A). In addition, 1×10⁷ VP of SFV-dcIL 12-Nb11 led to 28% long-term survivors compared to 0% with the same dose of SFV-dcIL 12.

### CITATION LIST

Rodriguez-Madoz J R, Prieto J, Smerdou C. Semliki forest virus vectors engineered to express higher IL-12 levels induce efficient elimination of murine colon adenocarcinomas. Mol Ther. 2005 July; 12 (1): 153-63. doi: 10.1016/j.ymthe.2005.02.011.
US 2010/0028330
Ballesteros-Briones M C, Martisova E, Casales E, Silva-Pilipich N, Buñuales M, Galindo J, Mancheño U, Gorraiz M, Lasarte J J, Kochan G, Escors D, Sanchez-Paulete A R, Melero I, Prieto J, Hernandez-Alcoceba R, Hervas-Stubbs S, Smerdou C. Short-Term Local Expression of a PD-L1 Blocking Antibody from a Self-Replicating RNA Vector Induces Potent Antitumor Responses. Mol Ther. 2019 Nov. 6; 27 (11): 1892-1905. doi: 10.1016/j.ymthe.2019.09.016. Epub 2019 Sep. 16.
Quetglas J I, Fioravanti J, Ardaiz N, Medina-Echeverz J, Baraibar I, Prieto J, Smerdou C, Berraondo P. A Semliki forest virus vector engineered to express IFNα induces efficient elimination of established tumors. Gene Ther. 2012 March; 19 (3): 271-8. doi: 10.1038/gt.2011.99. Epub 2011 Jul. 7.
Berglund P, Smerdou C, Fleeton M N, Tubulekas I, Liljeström P. Enhancing immune responses using suicidal DNA vaccines. Nat Biotechnol. 1998 June; 16 (6): 562-5. doi: 10.1038/nbt0698-562.
Liljeström P, Garoff H. Expression of proteins using Semliki Forest virus vectors. Curr Protoc Mol Biol. 2001
Smerdou C, Liljeström P. Two-helper RNA system for production of recombinant Semliki forest virus particles. J Virol. 1999; 73:1092-8
Silva-Pilipich N, Martisova E, Ballesteros-Briones M C, Hervas-Stubbs S, Casares N, González-Sapienza G, et al. Long-Term Systemic Expression of a Novel PD-1 Blocking Nanobody from an AAV Vector Provides Antitumor Activity without Toxicity. Biomedicines. 2020; 8 (12): 562
Dobin A, Davis C A, Schlesinger F, Drenkow J, Zaleski C, Jha S, et al. STAR: ultrafast universal RNA-seq aligner. Bioinformatics. 2013; 29:15-21
Silva-Pilipich N, Martisova E, Ballesteros-Briones M C, Hervas-Stubbs S, Casares N, González-Sapienza G, et al. Long-Term Systemic Expression of a Novel PD-1 Blocking Nanobody from an AAV Vector Provides Antitumor Activity without Toxicity. Biomedicines. 2020; 8 (12): 562
Liao Y, Smyth G K, Shi W. featureCounts: an efficient general purpose program for assigning sequence reads to genomic features. Bioinformatics. 2014; 30:923-30.
Harrow J, Frankish A, Gonzalez J M, Tapanari E, Diekhans M, Kokocinski F, et al. GENCODE: The reference human genome annotation for The ENCODE Project. Genome Res. 2012; 22:1760-74
Gentleman R C, Carey V J, Bates D M, Bolstad B, Dettling M, Dudoit S, et al. Bioconductor: open software development for computational biology and bioinformatics. Genome Biol. 2004; 5: R80.
Robinson M D, McCarthy D J, Smyth G K. edgeR: a Bioconductor package for differential expression analysis of digital gene expression data. Bioinformatics. 2010; 26:139-40
Ritchie M E, Phipson B, Wu D, Hu Y, Law C W, Shi W, et al. limma powers differential expression analyses for RNA-sequencing and microarray studies. Nucleic Acids Res. 2015; 43: e47-e47.
Subramanian A, Tamayo P, Mootha V K, Mukherjee S, Ebert B L, Gillette M A, et al. Gene set enrichment analysis: A knowledge-based approach for interpreting genome-wide expression profiles. Proc Natl Acad Sci. 2005; 102:15545-50.
Silva-Pilipich N, Lasarte-Cia A, Lozano T, Martin-Otal C, Lasarte J J, Smerdou C. Intratumoral electroporation of a self-amplifying RNA expressing interleukin-12 induces antitumor effects in mouse models of cancer. Molecular Therapy Nucleic Acids, 2022 Sep. 13; 29:387-399. Published online 2022 Jul. 20. doi: 10.1016/j.omtn.2022.07.020.
Xiang Z, Li J, Zhang Z, Cen C, Chen W, Jiang B, et al. Comprehensive Evaluation of Anti-PD-1, Anti-PD-L1, Anti-CTLA-4 and Their Combined Immunotherapy in Clinical Trials: A Systematic Review and Meta-analysis. Front Pharmacol. 2022; 13:883655.
Sun G, Liu H, Shi X, Tan P, Tang W, Chen X, et al. Treatment of patients with cancer using PD-1/PD-L1 antibodies: Adverse effects and management strategies (Review). Int J Oncol. 2022; 60.
Needleman and Wunsch, J. Mol. Biol. 48:443-453, 1970. DOI: 10.1016/0022-2836 (70) 90057-4.
Altschul et al., “Basic local alignment search tool”, 1990, J. Mol. Biol, v. 215, pages 403-410, 1990. DOI: 10.1016/S0022-2836 (05) 80360-2.
Grasselly C, Denis M, Bourguignon A, Talhi N, Mathe D, Tourette A, et al. The Antitumor Activity of Combinations of Cytotoxic Chemotherapy and Immune Checkpoint Inhibitors Is Model-Dependent. Front Immunol [Internet]. 2018; 9. Available from: https://www.frontiersin.org/article/10.3389/fimmu.2018.02100/full
Ngiow S F, Young A, Jacquelot N, Yamazaki T, Enot D, Zitvogel L, et al. A Threshold Level of Intratumor CD8+ T-cell PD1 Expression Dictates Therapeutic Response to Anti-PD1. Cancer Res [Internet]. 2015; 75:3800-11. Available from: https://aacrjournals.org/cancerres/article/75/18/3800/606326/A-Threshold-Level-of-Intratumor-CD8-T-cell-PD1
Komdeur F L, Singh A, van de Wall S, Meulenberg J J M, Boerma A, Hoogeboom B N, et al. First-in-Human Phase I Clinical Trial of an SFV-Based RNA Replicon Cancer Vaccine against HPV-Induced Cancers. Mol Ther. 2021; 29:611-25.
Markham A. Envafolimab: First Approval. Drugs. 2022; 82:235-40.
G. J. Lieschke, P. K. Rao, M. K. Gately, R. C. Mulligan, Bioactive murine and human interleukin-12 fusion proteins which retain antitumor activity in vivo., Nat. Biotechnol. 15 (1997) 35-40. https://doi.org/10.1038/nbt0197-35.

1. A single domain antibody (VHH or nanobody) comprising the following complementary determining regions (CDRs):
(i) CDR1 as set forth by SEQ ID NO: 1, CDR2 as set forth by SEQ ID NO: 2 and CDR3 as set forth by SEQ ID NO: 3; or
(ii) CDR1 as set forth by SEQ ID NO: 5, CDR2 as set forth by SEQ ID NO: 6 and CDR3 as set forth by SEQ ID NO: 7.

2. The nanobody according to claim 1, which
(i) is an anti-PD-L1 nanobody comprising CDR1 as set forth by SEQ ID NO: 1, CDR2 as set forth by SEQ ID NO: 2 and CDR3 as set forth by SEQ ID NO: 3, and is able to inhibit binding between PD-L1 and PD-1; or
(ii) is an anti-PD-1 nanobody comprising CDR1 as set forth by SEQ ID NO: 5, CDR2 as set forth by SEQ ID NO: 6 and CDR3 as set forth by SEQ ID NO: 7, and is able to inhibit binding between PD-L1 and PD-1.

3. The nanobody according to any one of claims 1-2, which blocks the binding between PD-L1 and PD-1 with half maximal inhibitory concentration (IC50) of 6 nM or lower.

4. The nanobody according to any one of claims 1-3, comprising or consisting of:
(i) a sequence as set forth in SEQ ID NO:4, or a sequence having at least 90%, or at least 95%, or at least 98%, or at least 99%, or 100% identity with SEQ ID NO: 4; or
(ii) a sequence as set forth in SEQ ID NO:8, or a sequence having at least 90%, or at least 95%, or at least 98%, or at least 99%, or 100% identity with SEQ ID NO: 8.

5. A nanobody conjugate comprising a nanobody according to any one of claims 1-4.

6. The nanobody conjugate according to the preceding claim, comprising at least one nanobody according to any one of claims 1-4 and an effector molecule.

7. The nanobody conjugate according to any one of claims 5-6, comprising two nanobodies as defined in any one of claims 1-4 fused to an immunoglobulin Fc domain.

8. The nanobody conjugate according to any one of claims 5-6, comprising at least one nanobody according to any one of claims 1-4 and IL-12.

9. A polynucleotide encoding for a nanobody as defined in anyone of claims 1-4 or for a nanobody conjugate as defined in any one of claims 5-8.

10. An expression vector comprising the polynucleotide as defined in the preceding claim.

11. The expression vector according to the preceding claim, which is a replication-defective alphavirus vector based on Semliki Forest virus (SFV).

12. A host cell comprising a polynucleotide as defined in claim 9 or an expression vector as defined in any one of claims 10-11.

13. A viral particle comprising an expression vector according to any one of claims 10-11 and SFV capsid and envelope proteins.

14. A pharmaceutical composition comprising a nanobody as defined in any one of claims 1-4, or a nanobody conjugate as defined in any one of claims 5-8, or an expression vector as defined in any one of claims 10-11, a host cell as defined in claim 12, or a viral particle as defined in claim 13, together with pharmaceutically acceptable excipients and/or carriers.

15. A nanobody as defined in any one of claims 1-4, or a nanobody conjugate as defined in any one of claims 5-8, or an expression vector as defined in any one of claims 10-11, a host cell as defined in claim 12, or a viral particle as defined in claim 13, or a pharmaceutical composition as defined in claim 14, for use in therapy, in particular, for the prevention and/or treatment of cancer.


SEQUENCE LISTING (31 sequences)

SEQ ID NO: 1 - protein, 8 aa, synthetic construct
GRSFNSNA

SEQ ID NO: 2 - protein, 9 aa, synthetic construct
ISATGSYTY

SEQ ID NO: 3 - protein, 17 aa, synthetic construct
AGSRTAALTTIMTLFNY

SEQ ID NO: 4 - protein, 125 aa, synthetic construct
AEVQLVESGGGLVQAGGSLRLSCAAAGRSFNSNAMGWVRQAPGKEREIVAVISATGSYTYYEDSVKGRFTISRDNSKNTVSLQMSSLSSEDTAVYYCAGSRTAALTTIMTLFNYWGQGTQVTVSS

SEQ ID NO: 5 - protein, 8 aa, synthetic construct
GHPDSSYS

SEQ ID NO: 6 - protein, 9 aa, synthetic construct
ISWSGAITH

SEQ ID NO: 7 - protein, 12 aa, synthetic construct
QADDPTSELNQY

SEQ ID NO: 8 - protein, 120 aa, synthetic construct
AQVQLVQSGGGSVQAGDSLRLSCAVSGHPDSSYSMAWFRQAPGKERELVALISWSGAITHYSDSVKDRFTISRDNAKNEMYLQMDSLKPEDTAVYFCQADDPTSELNQYWGQGTQVTVSS

SEQ ID NO: 9 - protein, 359 aa, synthetic construct
AEVQLVESGGGLVQAGGSLRLSCAAAGRSFNSNAMGWVRQAPGKEREIVAVISATGSYTYYEDSVKGRFTISRDNSKNTVSLQMSSLSSEDTAVYYCAGSRTAALTTIMTLFNYWGQGTQVTVSSPGPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 342 aa, synthetic construct
AQVQLVQSGGGSVQAGDSLRLSCAVSGHPDSSYSMAWFRQAPGKERELVALISWSGAITHYSDSVKDRFTISRDNAKNEMYLQMDSLKPEDTAVYFCQADDPTSELNQYWGQGTQVTVSSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 663 aa, synthetic construct
AEVQLVESGGGLVQAGGSLRLSCAAAGRSFNSNAMGWVRQAPGKEREIVAVISATGSYTYYEDSVKGRFTISRDNSKNTVSLQMSSLSSEDTAVYYCAGSRTAALTTIMTLFNYWGQGTQVTVSSGGGGSGGGGSGGGGSASMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 12 - protein, 681 aa, synthetic construct
PQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAEVQLVESGGGLVQAGGSLRLSCAAAGRSFNSNAMGWVRQAPGKEREIVAVISATGSYTYYEDSVKGRFTISRDNSKNTVSLQMSSLSSEDTAVYYCAGSRTAALTTIMTLFNYWGQGTQVTVSS

SEQ ID NO: 13 - protein, 658 aa, synthetic construct
AQVQLVQSGGGSVQAGDSLRLSCAVSGHPDSSYSMAWFRQAPGKERELVALISWSGAITHYSDSVKDRFTISRDNAKNEMYLQMDSLKPEDTAVYFCQADDPTSELNQYWGQGTQVTVSSGGGGSGGGGSGGGGSASMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 14 - protein, 676 aa, synthetic construct
PQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAQVQLVQSGGGSVQAGDSLRLSCAVSGHPDSSYSMAWFRQAPGKERELVALISWSGAITHYSDSVKDRFTISRDNAKNEMYLQMDSLKPEDTAVYFCQADDPTSELNQYWGQGTQVTVSS

SEQ ID NO: 15 - protein, 354 aa, synthetic construct
GQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAEVQLVESGGGLVQAGGSLRLSCAAAGRSFNSNAMGWVRQAPGKEREIVAVISATGSYTYYEDSVKGRFTISRDNSKNTVSLQMSSLSSEDTAVYYCAGSRTAALTTIMTLFNYWGQGTQVTVSS

SEQ ID NO: 16 - protein, 478 aa, synthetic construct
ATTMGPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSAEVQLVESGGGLVQAGGSLRLSCAAAGRSFNSNAMGWVRQAPGKEREIVAVISATGSYTYYEDSVKGRFTISRDNSKNTVSLQMSSLSSEDTAVYYCAGSRTAALTTIMTLFNYWGQGTQVTVSS

SEQ ID NO: 17 - protein, 349 aa, synthetic construct
GQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAQVQLVQSGGGSVQAGDSLRLSCAVSGHPDSSYSMAWFRQAPGKERELVALISWSGAITHYSDSVKDRFTISRDNAKNEMYLQMDSLKPEDTAVYFCQADDPTSELNQYWGQGTQVTVSS

SEQ ID NO: 18 - protein, 473 aa, synthetic construct
ATTMGPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSAQVQLVQSGGGSVQAGDSLRLSCAVSGHPDSSYSMAWFRQAPGKERELVALISWSGAITHYSDSVKDRFTISRDNAKNEMYLQMDSLKPEDTAVYFCQADDPTSELNQYWGQGTQVTVSS

SEQ ID NO: 19 - DNA, 375 bp, synthetic construct
  source 1..375: mol_type = other DNA
gccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctca

SEQ ID NO: 20 - DNA, 360 bp, synthetic construct
  source 1..360: mol_type = other DNA
gcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctca

SEQ ID NO: 21 - DNA, 8919 bp, synthetic construct
  source 1..8919: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccaactggggcctgaagctggtgttcttcgtgctgatcctgaagggcgtgcagtgcaccagtgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcaggtacctctagatacccctacgacgtgcccgactacgcctcctgacatatggggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 22 - DNA, 8904 bp, synthetic construct
  source 1..8904: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccaactggggcctgaagctggtgttcttcgtgctgatcctgaagggcgtgcagtgcaccagtgcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctcaggtacctctagatacccctacgacgtgcccgactacgcctcctgacatatggggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 23 - DNA, 9568 bp, synthetic construct
  source 1..9568: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccaactggggcctgaagctggtgttcttcgtgctgatcctgaagggcgtgcagtgcgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcacccgggcctagaggccccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaa

SEQ ID NO: 24 - DNA, 9517 bp, synthetic construct
  source 1..9517: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccaactggggcctgaagctggtgttcttcgtgctgatcctgaagggcgtgcagtgcgcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctcaggctgtaaaccttgcatatgtactgtcccagaagtgagctccgtcttcatctttccacccaagcctaaagacgtgctgaccattacactcactccaaaggtcacctgcgtggtcgtggacatcagcaaagacgatcctgaggtgcagttctcctggtttgtggacgatgtcgaagtgcacaccgctcagacacagcctagagaggaacagttcaacagcaccttcaggagcgtgagcgagctgccaattatgcatcaggactggctcaatgggaaggaattcaaatgcagggtgaacagcgcagcctttccagcacccatcgagaagaccatttccaagacaaaaggcagaccaaaagctccccaggtgtacaccatccctccacccaaggaacagatggcaaaggataaagtgtccctgacttgtatgattaccgacttctttccagaggatattacagtggaatggcagtggaatggtcagccagcagagaactataagaatacccagcccattatggacacagatgggtcttacttcgtgtatagtaagctgaacgtccagaaatctaattgggaagccggaaacacctttacatgtagtgtgctgcacgaggggctccataaccaccataccgaaaagagcctgtcccactctccaggaaaatgagggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaa

SEQ ID NO: 25 - DNA, 10513 bp, synthetic construct
  source 1..10513: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccaactggggcctgaagctggtgttcttcgtgctgatcctgaagggcgtgcagtgcgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcaggaggtggaggaagtggaggcggtgggtccggaggtggaggaagtgctagcatgtgggagctggaaaaggacgtgtacgtggtcgaggtggactggactccagatgctcccggcgaaaccgtgaacctgacatgcgacactcccgaggaagacgacatcacatggacttcagatcagcggcacggcgtgatcggcagcggaaagaccctgacaattactgtcaaagaattcctcgatgcaggacagtacacatgtcacaagggcggagagactctgagccactcccatctgctcctgcataagaaagagaacgggatctggagcactgaaattctgaagaacttcaagaacaagacctttctcaaatgcgaggcacctaattatagcggcaggttcacctgttcctggctggtgcagagaaacatggacctcaagtttaatatcaaaagctcctctagtccccctgattccagagccgtgacctgcggaatggcttctctgagtgcagagaaggtgacactcgaccagcgggattacgagaaatattcagtgagctgccaggaagacgtcacctgtccaacagccgaggaaactctgcccatcgagctcgcactggaagcccgacagcagaacaagtacgagaattattccacctctttctttattagggacatcattaagcctgatccacccaaaaacctgcagatgaagccactcaaaaattcacaggtggaggtcagctgggaataccccgacagttggtcaacacctcactcttatttcagtctgaagttctttgtgagaatccagcgcaagaaagagaagatgaaagaaaccgaggaagggtgcaaccagaagggtgcttttctggtggagaaaaccagtacagaggtccagtgcaagggcggcaacgtgtgtgtccaggcacaggacaggtactataattcaagctgtagcaagtgggcctgcgtgccatgtagggtcagatccggcggagggggttctggcggagggggtagtggcggagggggttcaagagtgattcctgtcagcggcccagcaagatgcctgagccagtccaggaatctcctgaagaccacagacgatatggtgaaaacagcccgcgagaagctgaaacactactcctgtactgctgaggacatcgatcatgaagacattacccgagatcagacttccaccctgaagacatgcctccccctggagctccataaaaacgaatcttgtctggctactcgcgagacctcctctactacccgagggtcttgcctgcctccacagaagaccagtctcatgatgacactgtgtctcggttctatctacgaggacctgaaaatgtatcagacagaattccaggccattaatgccgctctccagaaccacaatcatcagcagatcattctggacaagggaatgctcgtggctatcgatgagctgatgcagtctctcaaccacaatggcgaaacactgagacagaagccccctgtgggagaggctgatccctaccgcgtcaagatgaaactgtgtatcctcctgcatgccttcagcacaagagtggtcactattaacagagtgatggggtatctgagttcagcctaagcggccgcatatgatatcggatccatcggatcccgggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 26 - DNA, 10666 bp, synthetic construct
  source 1..10666: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccgggcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggtggaggaagtggaggcggtgggagtggaggtggcggatccgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcacatatgaatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgatagtcgcgacccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 27 - DNA, 10498 bp, synthetic construct
  source 1..10498: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccaattggggtctcaaactcgtgttcttcgtgctcatcctcaagggtgtccagtgtgctcaggtccagctcgtccagtccgggggtggctccgtgcaggccggagactctctgaggctgagctgcgccgtgagcggccacccagatagctcctactctatggcctggttcaggcaggctcccggaaaggagagggagctggtggccctgatctcctggtctggcgctatcacccactacagcgactccgtgaaggatcggttcacaatctctcgcgacaacgccaagaacgagatgtacctgcagatggacagcctgaagcctgaggataccgccgtgtacttttgtcaggctgacgatccaacaagcgagctgaaccagtactggggacagggcacccaggtgacagtgtctagcggaggtggaggaagtggaggcggtgggtccggaggtggaggaagtgctagcatgtgggagctggaaaaggacgtgtacgtggtcgaggtggactggactccagatgctcccggcgaaaccgtgaacctgacatgcgacactcccgaggaagacgacatcacatggacttcagatcagcggcacggcgtgatcggcagcggaaagaccctgacaattactgtcaaagaattcctcgatgcaggacagtacacatgtcacaagggcggagagactctgagccactcccatctgctcctgcataagaaagagaacgggatctggagcactgaaattctgaagaacttcaagaacaagacctttctcaaatgcgaggcacctaattatagcggcaggttcacctgttcctggctggtgcagagaaacatggacctcaagtttaatatcaaaagctcctctagtccccctgattccagagccgtgacctgcggaatggcttctctgagtgcagagaaggtgacactcgaccagcgggattacgagaaatattcagtgagctgccaggaagacgtcacctgtccaacagccgaggaaactctgcccatcgagctcgcactggaagcccgacagcagaacaagtacgagaattattccacctctttctttattagggacatcattaagcctgatccacccaaaaacctgcagatgaagccactcaaaaattcacaggtggaggtcagctgggaataccccgacagttggtcaacacctcactcttatttcagtctgaagttctttgtgagaatccagcgcaagaaagagaagatgaaagaaaccgaggaagggtgcaaccagaagggtgcttttctggtggagaaaaccagtacagaggtccagtgcaagggcggcaacgtgtgtgtccaggcacaggacaggtactataattcaagctgtagcaagtgggcctgcgtgccatgtagggtcagatccggcggagggggttctggcggagggggtagtggcggagggggttcaagagtgattcctgtcagcggcccagcaagatgcctgagccagtccaggaatctcctgaagaccacagacgatatggtgaaaacagcccgcgagaagctgaaacactactcctgtactgctgaggacatcgatcatgaagacattacccgagatcagacttccaccctgaagacatgcctccccctggagctccataaaaacgaatcttgtctggctactcgcgagacctcctctactacccgagggtcttgcctgcctccacagaagaccagtctcatgatgacactgtgtctcggttctatctacgaggacctgaaaatgtatcagacagaattccaggccattaatgccgctctccagaaccacaatcatcagcagatcattctggacaagggaatgctcgtggctatcgatgagctgatgcagtctctcaaccacaatggcgaaacactgagacagaagccccctgtgggagaggctgatccctaccgcgtcaagatgaaactgtgtatcctcctgcatgccttcagcacaagagtggtcactattaacagagtgatggggtatctgagttcagcctaagcggccgcatatgatatcggatccatcggatcccgggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 28 - DNA, 10654 bp, synthetic construct
  source 1..10654: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccgggcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggtggaggaagtggaggcggtgggagtggaggtggcggatccgcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctcatgacatatgaatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgatagtcgcgacccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 29 - DNA, 11222 bp, synthetic construct
  source 1..11222: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggtggaggaagtggaggcggtgggtccggaggtggaggaagtgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcatgaggcgcgcccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctggacccgccacaaccatgggtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactctggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatctggaggtggaggaagtggaggcggtgggtccggcggtggaggatccgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcatgacatatggggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 30 - DNA, 11192 bp, synthetic construct
  source 1..11192: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggtggaggaagtggaggcggtgggtccggaggtggaggaagtgcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctcatgaggcgcgcccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctggacccgccacaaccatgggtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactctggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatctggaggtggaggaagtggaggcggtgggtccggcggtggaggatccgcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctcatgacatatggggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa

SEQ ID NO: 31 - DNA, 11207 bp, synthetic construct
  source 1..11207: mol_type = other DNA
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctgggcccggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaggtggaggaagtggaggcggtgggtccggaggtggaggaagtgccgaggtgcagctggtggagtctgggggaggattggtgcaggctggtggctctctgagactctcctgtgcagccgctggacgcagcttcaattccaatgccatgggctgggtccgccaggctccagggaaggagcgtgagattgtcgcggttattagtgcgacaggcagttatacatactatgaagactccgtgaagggccgattcaccatttccagagacaactccaagaacacggtttctctgcaaatgagcagtttgagttctgaggatacggccgtttattactgtgccggttcgcgaacagcggctttaaccacaataatgactttgtttaactactggggccaggggacccaggtcactgtctcctcatgaggcgcgcccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccttggccgttgcaggccactccggtggctcccgtcgtcaattttgaccttcttaagcttgcgggagacgtcgagtccaaccctggacccgccacaaccatgggtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactctggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatctggaggtggaggaagtggaggcggtgggtccggcggtggaggatccgcccaggtgcagctggtgcagtctgggggaggatcggtgcaggctggggactctctgagactctcctgtgcagtttctggacaccccgacagttcttattccatggcctggttccgccaggctccagggaaggagcgggaattagtagcgcttatcagctggagcggtgctatcacacactattcagattccgtgaaggaccgattcaccatctccagagacaacgccaagaacgaaatgtatctacaaatggacagcctcaaacctgaggacacggccgtttatttctgtcaagcggacgaccccacttcagagcttaatcagtactggggccaggggacccaggtcactgtctcctcatgacatatggggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaa